(19) United States
(12) Patent Application Publication
(10) Pub. No.: US 2024/0294439 A1
(43) Pub. Date: Sep. 5, 2024

(54) USE OF BRINE AND NATURAL POZZOLANS IN A METHOD OF MAKING COMPOSITIONS TO PROMOTE MARINE-LIFE

(71) Applicant: Partanna Global, Inc., Rancho Santa Fe, CA (US)

(72) Inventors: Sam Franklin Marshall, Hidden Hills, CA (US); Kevin MacDonald, Rancho Santa Fe, CA (US)

(21) Appl. No.: 18/424,515

(22) Filed: Jan. 26, 2024

Related U.S. Application Data
(60) Provisional application No. 63/481,910, filed on Jan. 27, 2023.

(51) Int. Cl. C04B 40/02; C02F 1/52; C04B 14/14; C04B 22/06; C04B 22/10; C04B 22/14; C04B 28/04; C04B 28/08; C04B 28/10; C04B 28/14; C04B 40/00; C12N 1/12; C02F 103/08; C02F 103/34; C04B 103/00

(52) U.S. Cl. CPC C04B 40/0231; C02F 1/5245; C04B 14/14; C04B 22/064; C04B 22/066; C04B 22/10; C04B 22/148; C04B 28/04; C04B 28/08; C04B 28/105; C04B 28/14; C04B 40/0046; C04B 40/0071; C12N 1/12; C02F 2103/08; C02F 2103/34; C04B 2103/0001

(57) ABSTRACT

The present disclosure relates to methods that transform high salinity brine into a non-toxic, CO₂-absorbing cementitious aggregate composition and a lower salinity water component which can each be recycled to the desalination facility, further processed to produce fresh water, and/or added to a body of water and without harming the local flora and fauna. The present disclosure provides methods comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a cementitious aggregate composition and a reduced salinity water component. In some case, an activator, as disclosed herein, is added to the combination. The present disclosure further a cementitious aggregate compositions and shaped cementitious compositions, each obtained by herein-disclosed methods.

### CROSS-REFERENCE

This application claims the benefit of U.S. Provisional Application No. 63/481,910 filed Jan. 27, 2023, the entirety of which are hereby incorporated by reference herein.

### BACKGROUND

Access to freshwater in many areas of the world is a growing environmental problem. To address the shortfall of potable water, desalination facilities convert seawater into drinkable water; however, these facilities produce as byproducts a wastewater that is concentrated in salts, minerals, and other chemical residues, known as brine. There are hundreds of millions of gallons of brine wastewater dumped into the oceans each day from desalination facilities around the globe. This high salinity wastewater not only has no economic value, but also has adverse effects on marine ecosystems. Dumping brine wastewater disturbs the local water and sediment, at least, by introducing a multi-component waste, increasing the local salinity, and increasing the temperature; these changes are harmful to the local flora and fauna. There remains an unmet need for transforming brine wastewater from desalination facilities into products that are, at least, not harmful to aquatic life and which may be beneficial to them.

### BRIEF SUMMARY

The present disclosure relates to methods that transform high salinity brine into a non-toxic, CO₂-absorbing aggregate cementitious composition and a lower salinity water component which can be recycled through the desalination plant, to create additional drinking water, or added to a body of water and without harming the local flora and fauna.

An aspect of the present disclosure is a method comprising steps of: (a) combining (i) a high salinity brine, (ii) CO2, and (iii) a natural pozzolan, a man-made pozzolan, and/or a latently hydraulic material; and, optionally, an activator (which may be any sodium, lithium or potassium salt or hydroxide and any alkali or alkali earth metal ions and including Mg(OH)2. MgO, Ca(OH)₂, CaCO₃, Al₂(SO₄)₃, and/or CaO) and (b) permitting the combination obtained in step (a) to persist under conditions sufficient to transform the combination into a cementitious aggregate composition and a reduced salinity water component.

In an aspect, the present disclosure provides an in-line conversion method comprising steps of: (a) combining (i) a high salinity brine, (ii) CO₂, and (iii) a natural pozzolan, a man-made pozzolan, and/or a latently hydraulic material; and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO) and (b) permitting the combination obtained in step (a) to persist under conditions sufficient to transform the combination into a cementitious aggregate composition and a reduced salinity water component, wherein the conditions in step (b) preferably comprises applied pressure and wherein steps (a) and/or (b) occur in a pipeline.

Another aspect of the present disclosure is an in-line conversion method comprising steps of: (a) combining (i) a high salinity brine, (ii) CO₂, and (iii) a pozzolan and/or a latently hydraulic material; and (b) applying pressure to the combination obtained in step (a) for an amount and duration sufficient to transform the combination into a cementitious aggregate composition and a reduced salinity water component, wherein the conditions in step (b) comprise applied pressure and wherein steps (a) and/or (b) occur in a pipeline.

An additional aspect of the present disclosure is an in-line conversion method comprising steps of: (a) combining (i) a high salinity brine, (ii) CO₂, and (iii) a pozzolan and/or a latently hydraulic material; (b) allowing the combination obtained in step (a) to persist for duration sufficient to transform the combination into a cementitious aggregate composition and a reduced salinity water component, wherein steps (a) and/or (b) occur in a pipeline.

Another aspect of the present disclosure is an in-line conversion method comprising steps of: (a) combining (i) a high salinity brine, (ii) CO₂, (iii) a pozzolan and/or a latently hydraulic material, and (iv) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); and (b) applying pressure to the combination obtained in step (a) for an amount and duration sufficient to transform the combination into a cementitious aggregate composition and a reduced salinity water component, wherein the conditions in step (b) comprise applied pressure and wherein steps (a) and/or (b) occur in a pipeline.

A further aspect of the present disclosure is a tank precipitation method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a cementitious aggregate composition and a reduced salinity water component, wherein the conditions in step (b) does not comprise applied pressure and wherein steps (a) and/or (b) occur in a tank or basin. In some cases, the conditions sufficient to transform the combination into a cementitious aggregate composition and a reduced salinity water component in step (b) comprise time and heat.

In another aspect, the present disclosure provides a tank precipitation method comprising steps of: (a) combining (i) a high salinity brine, (ii) CO₂, and (iii) a natural pozzolan, a man-made pozzolan, and/or a latently hydraulic material; and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO) and (b) permitting the combination obtained in step (a) to persist under conditions sufficient to transform the combination into a cementitious aggregate composition and a reduced salinity water component, wherein the conditions in step (b) does not comprise applied pressure and wherein steps (a) and/or (b) occur in a tank or basin. In some cases, the conditions sufficient to transform the combination into a cementitious aggregate composition and a reduced salinity water component in step (b) comprise time and heat.

In various tank precipitation methods, an activator is preferably included in the combination.

In combinations comprising latently hydraulic material, an activator is preferably included in the combination.

In some cases, heat is applied to the mixture and/or the mixture is heated via an additional heat source.

In some embodiments, the reduced salinity water component is further processed to extract sodium to produce additional fresh water. In addition to or in combination with or after the above-described processes, a chloralkali process (as illustrated in FIG. 5) can more efficiently be performed, since, at least in part, because the sulphates and chlorides have already been removed. The extracted sodium may be isolated into a pure sodium product.

A further aspect of the present disclosure is a method for providing substrate for aquatic flora and/or fauna attachment, the method comprising depositing a herein-disclosed cementitious aggregate composition into a natural or artificial body of water which comprises aquatic flora and/or fauna in need of an attachment.

Another aspect of the present disclosure is a cementitious aggregate composition obtained by any herein-disclosed method. The cementitious aggregate composition be used in any way that another cementitious aggregates can be used.

An additional aspect of the present disclosure is a shaped cementitious composition obtained by any herein-disclosed method. The shaped cementitious aggregate composition be used in any way that any cement can be used.

In an aspect, the present disclosure provides a method for providing substrate for aquatic flora and/or fauna attachment, the method comprising depositing any herein-disclosed shaped cementitious composition into a natural or artificial body of water which comprises aquatic flora and/or fauna in need of an attachment.

In another aspect, the present disclosure provides a cementitious aggregate composition comprising more CO₂ per gram and/or calcium, silicon, aluminum, magnesium and/or iron per gram than a standard cement.

In a further aspect, the present disclosure provides a cementitious aggregate composition comprising more numerous pores per unit volume and/or larger average pores than a standard cement.

Any composition or method disclosed herein is applicable to any herein-disclosed composition or method. In other words, any aspect or embodiment described herein can be combined with any other aspect or embodiment as disclosed herein.

### BRIEF DESCRIPTION OF THE DRAWINGS

FIG. 1 is a schematic illustrating steps of herein-disclosed methods.

FIG. 2 is a photograph showing an illustrative cementitious aggregate composition obtained by herein-disclosed methods.

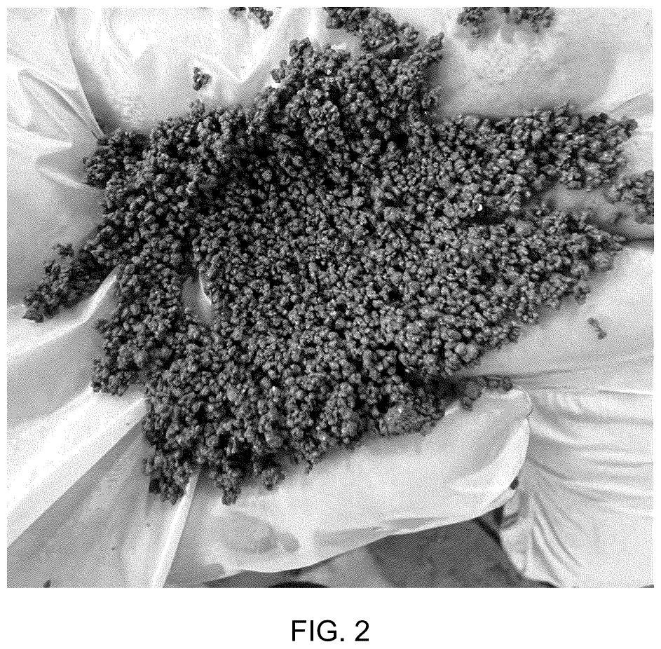

FIG. 3 is a schematic illustrating the process of in-line conversion. Also shown are precipitated compounds resulting from the method.

FIG. 4 is a schematic illustrating the process of tank precipitation.

FIG. 5 is a schematic for removing sulphates and sodium from a high salinity brine.

### DETAILED DESCRIPTION

### Introduction

The present disclosure relates to methods that transforms a high salinity brine, which may be a byproduct from a desalination facility's conversion of seawater into drinkable water, into (1) a non-toxic, CO₂-absorbing cementitious aggregate composition and (2) a lower salinity water component. The lower salinity water can be recycled through the desalination plant, to create additional drinking water, or added to a body of water and without harming the local flora and fauna; however, in some cases, the lower salinity water component is fresh water. And, the cementitious aggregate composition can be added to a body of water to promote coral growth due, in part, to the chemical composition of the material.

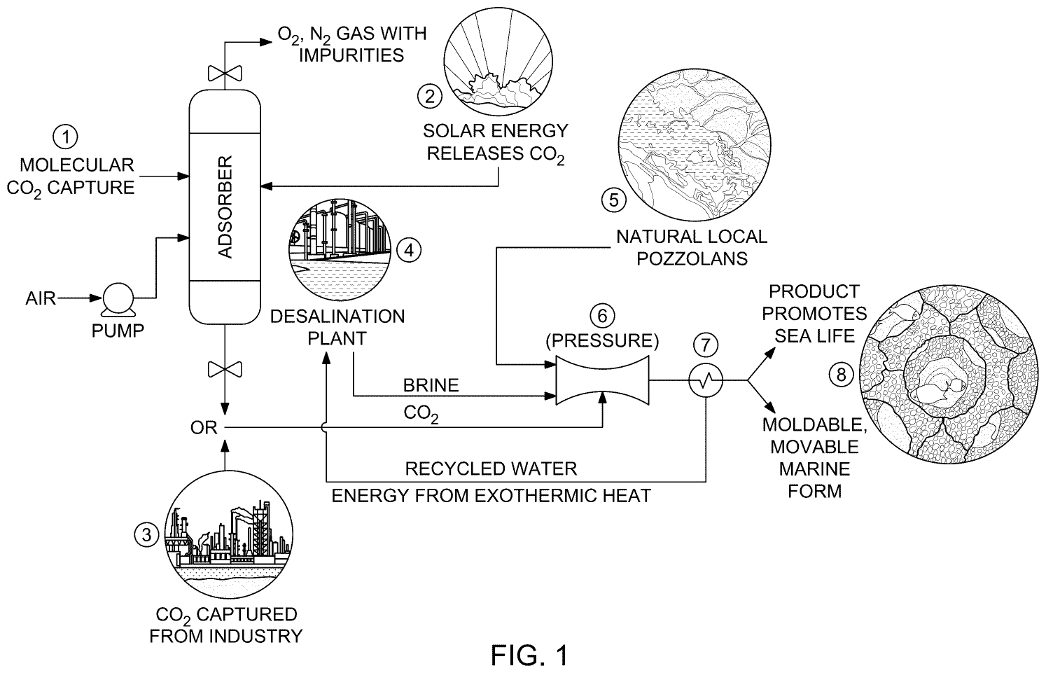

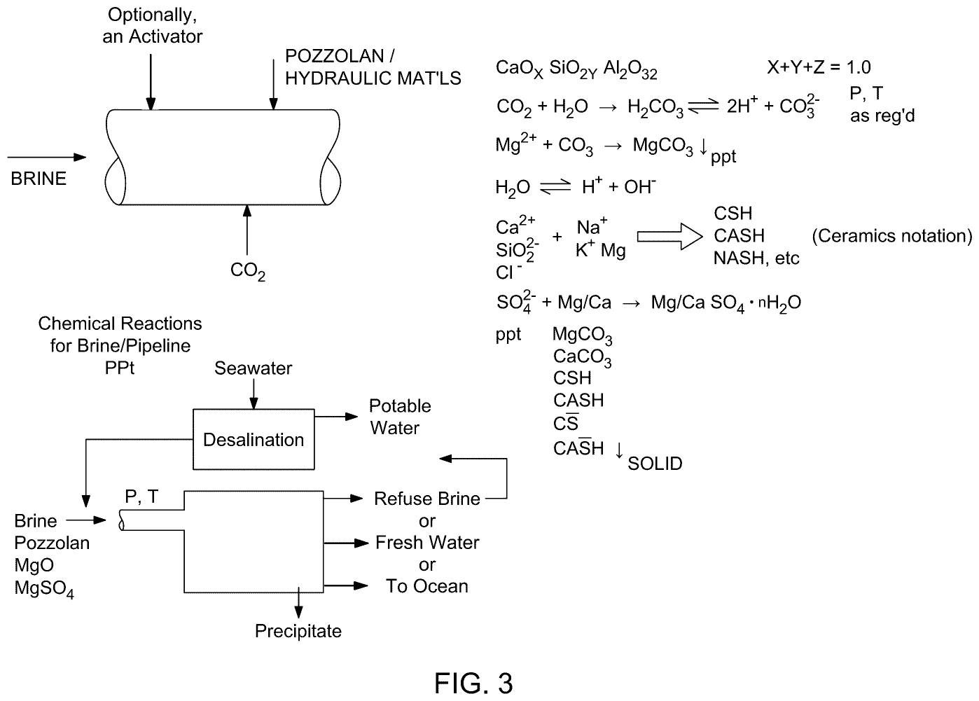

Elements of an illustrative inline conversion process of the present disclosure are shown in FIG. 1. CO₂ is captured through industrial processes (3) or from direct air molecular capture (1). In a brine discharge pipeline or pipeline, the CO₂ is combined with a high salinity brine from a desalination facility (4) along with dry-mix composition comprising a natural pozzolan, a man-made pozzolan, and/or a latently hydraulic material; and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO). The inline conversion process takes advantage of the existing in-line pressure and volume of the brine in the outlet pipe. The efficiency of converting the dissolved salts takes advantage of magnifying the existing forces (pressure) by utilizing a series of pipe reducers and/or other methodologies that do not require addition energy inputs; in some cases, additional energy inputs are provided. Once pressure is applied to the combination, a cementitious aggregate composition is created (8) and a reduced salinity water component is created. The cementitious aggregate composition can be captured by straining or precipitation to obtain an isolated product. The cementitious aggregate composition can directly be used as a substrate for attachment by aquatic life or can be molded into defined shapes and which then can be used as a substrate for attachment by aquatic life. And, the reduced salinity water component can be recycled to the desalination facility or may be is added to a natural or artificial body of water where it will cause less harm to local aquatic life than the high salinity brine from the desalination facility. Moreover, the cementitious aggregate composition be used in ways that any aggregates can be used. Finally, exothermic heat produced from the pressure and chemical reactions (7) which can be captured and used for energy in the desalination facility, or elsewhere, using well-established methodologies. See also FIG. 3 is another schematic illustrating the process of in-line conversion.

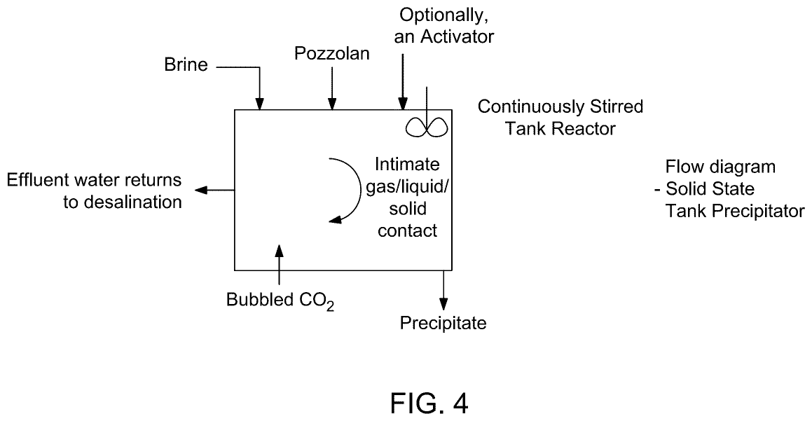

Elements of an illustrative tank conversion process of the present disclosure are shown in FIG. 4. CO₂ is captured through industrial processes or from direct air molecular capture. In tank or basin, the CO₂ is combined with a high salinity brine from a desalination facility (4) along with dry-mix composition comprising a natural pozzolan, a man-made pozzolan, and/or a latently hydraulic material; and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO). The combination is gently stirred or agitated to form insolvable precipitates with the salts (called flocs), which can settle and be separated from the liquid mixture. Ultimately, the cementitious aggregate composition can be captured by straining or precipitation to obtain an isolated product, which can be used as described for other methods of the present disclosure. Notably, the tank conversion process removes sulphates and chlorides from the brine but leaves the sodium. The remaining sodium can then be isolated by an efficient process, at least in part, since other minerals have already been removed and the isolated sodium will be of high purity since it will lack the other minerals. Further, after sodium isolation, the process permits production of additional fresh water.

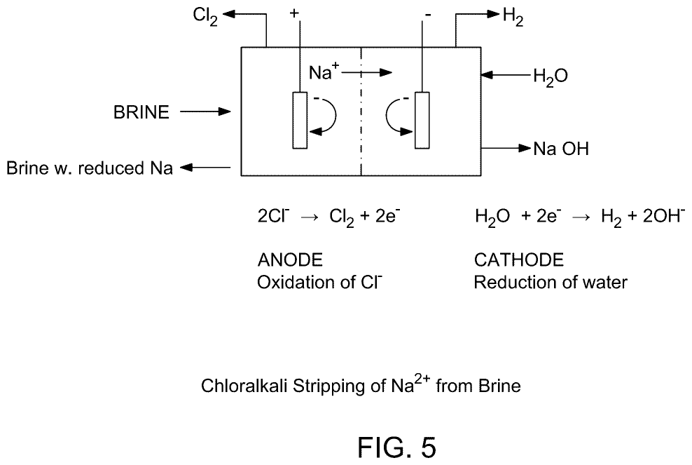

In addition to or in combination with or after the above-described processes, a chloralkali process (as illustrated in FIG. 5) can more efficiently be performed, since, at least in part, because the sulphates and chlorides have already been removed. Again, this permits creation of a higher purity isolated sodium and more fresh water.

In short, the present disclosure provides methods for using or “recycling” waste brine from a desalination facility (for example) while producing a lower salinity water component and a cementitious aggregate composition. The cementitious aggregate composition may be used as a substrate for attachment by aquatic flora and/or fauna. And, the lower salinity water component may be used to isolate pure sodium and to produce additional fresh water. In some cases, the lower salinity water component is fresh water.

Notably, the methods capture free CO₂ pollutants, thereby reducing its effect on climate change and providing an opportunity for collection of carbon credits.

To date, a desalination facility typically uses established reverse osmosis or membrane technologies to purify water because the process of converting dissolved salts into solids under pressure is not as efficient for large-scale water treatment when compared with these membrane methodologies.

However, given the significant negative effects on marine health, sea life, including coral, from discharging the high salinity brine derived from a desalination facility into the seas and oceans, global calls for mitigation of this issue are growing and are needed. On average, desalination facilities generally produce a high salinity brine discharge that is approximately 1.5 to 2.5 times the volume of the treated freshwater produced. For example, a large-scale desalination facility with a capacity of 100 million gallons per day (MGD) might produce a brine discharge of approximately 150 to 250 MGD.

As the concentration of salts and other elements within the discharged high salinity brine are far more significant, the process of combining pressure and added natural and recycled ingredients is efficient and effective. The present disclosure describes methods in which the pressure needed pressure to treat the high salinity brine discharged from desalination facility is less than half than what is needed to treat raw seawater-from 250 atmospheres or roughly 3,700 psi to roughly 100 atmospheres or 1,700 psi. This is because the solubility of salts generally decreases as the concentration of the solution increases.

When mixture of (i) a natural pozzolan, a man-made pozzolan, and/or a latently hydraulic material; and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); (ii) CO₂; and (iii) a high salinity brine are combined under this pressure, “flash reactions” or precipitation occurs. The benefit of this method is: (1) the generation of fresh water, in addition to the fresh water produced by the desalination facility, (2) the creation of a moldable cementitious product, (3) the creation of usable aggregate for use in cement products, (4) highly pure isolatable sodium, and, perhaps most importantly, (4) the removal of CO2 from the atmosphere.

The herein-described methods can follow, at least, two routes: in line conversion under pressure and tank precipitation.

In some cases, the process of in-line conversion takes advantage of the existing in-line pressure and volume of the high salinity brine in the outlet pipe from a desalination facility. The efficiency of converting dissolved salts relies on magnifying the existing forces (pressure) within the outlet pipe by utilizing a series of pipe reducers. The novel use of the reducers relies on the existing energy of the discharge without needing additional energy. This pressure is generated due to the resistance offered by the smaller diameter, causing an increase in fluid velocity and, subsequently, pressure.

In various cases, the process of tank precipitation makes use of chemical precipitation. Chemicals such as lime (calcium hydroxide) or alum (aluminum sulfate) can be added to the water to form insoluble precipitates with the salts, which can then settle and be separated from the water. This method is often used in municipal wastewater treatment and industrial processes. The “novel mix” of the present disclosure (comprising a natural pozzolan, a man-made pozzolan, and/or a latently hydraulic material and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO), CO₂, and high salinity brine precipitate in the tank which can be collected through processes of sedimentation, filtration, and/or clarification.

A general overview of the tank-based precipitation involves the following steps. 1. Mixing: The novel mix is combined with the high salinity brine (which contains dissolved salts-or activators for this purpose). These ingredients react with the salts to form insoluble precipitates or cementitious particles or flocs. 2: Flocculation: The mixture is gently stirred or agitated to promote the formation of larger particles called flocs. The flocs are composed of the precipitated salts and other impurities and ingredients. 3. Sedimentation: The mixture is allowed to settle in large tanks or basins, allowing the flocs to settle to the bottom due to gravity. This process is known as sedimentation or clarification. The settled flocs form a layer of sediment at the bottom of the tank. 4. Sediment removal: The sediment layer is periodically removed from the bottom of the tank using equipment such as sediment scrapers, screens and/or pumps. 5. Filtration (if required): In some cases, after sedimentation, the water may undergo additional filtration processes to remove any remaining suspended solids or fine particles that may have not settled during sedimentation. The time required for this precipitation process, including sedimentation removal, can vary depending on several factors such as the type and concentration of salts, the dosage and type of ingredients used, the temperature, and the design of the sedimentation tanks. In some cases, the process takes a few hours to complete. The goal is to allow sufficient time for the cementitious sediment to form, settle, and compact at the bottom of the tank. The sedimentation process can be enhanced by optimizing various factors, including the use of coagulants and flocculants, controlling the flow rate and hydraulic loading, and ensuring proper tank design and operation. These measures aim to maximize the removal efficiency of suspended solids and achieve the desired water quality.

In the methods of the present disclosure, magnesium oxide (MgO) can be used as a coagulant or flocculant in water treatment processes to create flocs. When added to water, MgO can undergo hydrolysis, generating hydroxyl ions (OH—) that can react with dissolved salts and impurities, leading to the formation of insoluble precipitates or flocs. The hydroxyl ions generated from the hydrolysis of MgO can neutralize the charges on suspended particles, destabilizing them and promoting their aggregation into larger flocs. These flocs can then settle more easily during the sedimentation process, facilitating their removal from the water. MgO is sometimes used as a coagulant or flocculant in various water treatment applications, including the treatment of industrial wastewater, municipal water, and wastewater treatment plants. It can be particularly effective in treating water with high levels of turbidity, color, or dissolved solids. However, in combination with the novel mix, it helps forms usable cementitious materials and/or aggregates.

Volcanic ash (natural pozzolan) can be used to create flocs. Volcanic ash contains various minerals and clays that have flocculation properties. When volcanic ash is added to water, it can adsorb onto suspended particles, destabilizing them and promoting the formation of flocs. The specific composition of volcanic ash can vary depending on the volcanic source, but it often contains minerals like zeolites, bentonite, or montmorillonite, which have high cation exchange capacities and surface charges. These properties allow volcanic ash to attract and bind with suspended particles, leading to the formation of larger flocs. Volcanic ash can be particularly effective where the removal of fine suspended particles or colloids is desired. It can be used in processes such as coagulation, flocculation, or sedimentation, where the ash is added to the water and mixed to facilitate the formation of flocs. When a natural pozzolan (e.g., a volcanic ash) or a man-made pozzolan (e.g., GGBFS slag), MgO, and CO₂ gas are used in combination with the high salinity brine, to creates hard cementitious flocs or sediment. Slag is a byproduct of the iron and steel manufacturing process and is commonly used as a supplementary cementitious material in concrete production. It contains reactive compounds such as calcium oxide (CaO) and silica (SiO₂) that can react with water and form cementitious compounds.

CO₂ gas is used to promote carbonation in cementitious materials. When CO₂ reacts with calcium hydroxide (Ca(OH)2), which is commonly present in cementitious systems, it can form calcium carbonate (CaCO₃). This reaction contributes to the hardening of the flocs or sediment, making them more durable and cementitious in nature.

Thus, by combining a natural pozzolan, a man-made pozzolan, and/or a latently hydraulic material, MgO or CaO or Mg(OH)₂, and CO₂ gas, it is possible to create a cementitious floc or sediment system where the slag provides the cementitious properties, MgO contributes to the flocculation process, and CO₂ gas promotes carbonation and hardening.

In addition, by adding CO₂ to water—it will lower the pH, which improves the solubility of certain salts and impurities, causing them to precipitate and form flocs—making the process more efficient. However, this effect is not directly related to CO₂ acting as a flocculant itself. Instead, it is the change in pH that triggers the precipitation of specific compounds.

CO₂ is used in some instances for pH adjustment or as a means of controlling alkalinity. By adjusting the pH, certain reactions can occur that facilitate the formation of flocs, but CO₂ is not the primary flocculant agent in these cases.

In the case of in line conversions, applying pressure to the brine outlet pipe from a desalination plant creates the feasibility of using slag, MgO, and CO₂ gas to create hard cementitious flocs in that specific context.

In various embodiments, a mixture which is transformed into a cementitious aggregate composition and a reduced salinity water component, may comprise one of the following combinations:
(1) brine, (2) CO₂, and (3) a natural pozzolan;
(1) brine, (2) CO₂, (3) a natural pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a man-made pozzolan;
(1) brine, (2) CO₂, (3) a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a latently hydraulic material;
(1) brine, (2) CO₂, (3) a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan and a man-made pozzolan;
(1) brine, (2) CO₂, (3) a natural pozzolan and a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan and a latently hydraulic material;
(1) brine, (2) CO₂, (3) a natural pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a man-made pozzolan and a latently hydraulic material;
(1) brine, (2) CO₂, (3) a man-made pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan, a man-made pozzolan, and a latently hydraulic material; or
(1) brine, (2) CO₂, (3) a natural pozzolan, a man-made pozzolan, and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO).

In various tank precipitation methods, an activator is preferably included in the combination.

In combinations comprising latently hydraulic material, an activator is preferably included in the combination.

The methods and compositions of the present disclosure further provide:
1. Fresh water generation: The processes effectively remove salts and impurities from the brine, producing fresh water as a byproduct. This is beneficial in areas where freshwater resources are scarce and/or where there is a demand for additional freshwater supply—and at little to no additional cost.
2. Carbon removal: The use of CO₂ gas in the process contributes to carbon removal from the atmosphere, and possible carbon credits. The amount depends on the specific carbon accounting mechanisms and standards in place, as well as the volume of CO₂ captured and utilized.
3. Market demand: Creation of much needed cementitious compositions, including aggregates.
4. Significant Reduction in Brine Pollution: This can reduce harm done to local flora and fauna.
5. High Pure Sodium: This can be isolated from residual water and using a highly efficient process.
5. Cost-effectiveness: The economic viability of the process is a critical advantage for its commercial viability. The costs associated with the relatively little needed additional infrastructure and equipment, are greatly outweighed by the benefits of additional freshwater production, much needed cementitious composition, and CO₂ absorption and offsets (including carbon credits).

### Methods

An aspect of the present disclosure is a method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a cementitious aggregate composition and a reduced salinity water component.

Another aspect of the present disclosure is an in-line conversion method comprising steps of: (a) combining (i) a high salinity brine, (ii) CO₂, and (iii) a pozzolan and/or a latently hydraulic material; and (b) applying pressure to the combination obtained in step (a) for an amount and duration sufficient to transform the combination into a cementitious aggregate composition and a reduced salinity water component, wherein the conditions in step (b) comprise applied pressure and wherein steps (a) and/or (b) occur in a pipeline.

An additional aspect of the present disclosure is an in-line conversion method comprising steps of: (a) combining (i) a high salinity brine, (ii) CO₂, and (iii) a pozzolan and/or a latently hydraulic material; (b) allowing the combination obtained in step (a) to persist for duration sufficient to transform the combination into a cementitious aggregate composition and a reduced salinity water component, wherein steps (a) and/or (b) occur in a pipeline.

Another aspect of the present disclosure is an in-line conversion method comprising steps of: (a) combining (i) a high salinity brine, (ii) CO₂, (iii) a pozzolan and/or a latently hydraulic material, and (iv) an activator; and (b) applying pressure to the combination obtained in step (a) for an amount and duration sufficient to transform the combination into a cementitious aggregate composition and a reduced salinity water component, wherein the conditions in step (b) comprise applied pressure and wherein steps (a) and/or (b) occur in a pipeline.

A further aspect of the present disclosure is a tank precipitation method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a cementitious aggregate composition and a reduced salinity water component, wherein the conditions in step (b) does not comprise applied pressure and wherein steps (a) and/or (b) occur in a tank or basin.

In various tank precipitation methods, an activator is preferably included in the combination.

In combinations comprising latently hydraulic material, an activator is preferably included in the combination.

In some embodiments, the high salinity brine is treated after step (a) to reduce the amount of sodium and chloride using a method known in the art (e.g., via a chloralkali process).

In some embodiments, the reduced salinity water component is further processed to extract sodium to produce additional fresh water. In addition to or in combination with or after the above-described processes, a chloralkali process (as illustrated in FIG. 5) can more efficiently be performed, since, at least in part, because the sulphates and chlorides have already been removed. The extracted sodium may be isolated into a pure sodium product.

In several embodiments, the high salinity brine, CO₂, and pozzolan and/or a latently hydraulic material are combined simultaneously.

In some embodiments, the high salinity brine, CO₂, and pozzolan and/or a latently hydraulic material are combined sequentially with the high salinity brine and CO₂ combined first.

In various embodiments, the high salinity brine, CO₂, and pozzolan and/or a latently hydraulic material are combined sequentially with the CO₂ and the pozzolan combined first.

In numerous embodiments, the high salinity brine, CO₂, and pozzolan and/or a latently hydraulic material are combined sequentially with the high salinity brine and the pozzolan combined first.

In additional embodiments, the high salinity brine is obtained from a desalination facility, is natural seawater, or an industrial brine.

In several embodiments, the salt concentration of the high salinity brine is greater than or about equal to the salt concentration of seawater. In some cases, the salt comprises sodium chloride.

In embodiments, the salt concentration of the reduced salinity water component is less than or about equal to the salt concentration of seawater. In some cases, the salt comprises sodium chloride.

In some embodiments, the salt concentration of the reduced salinity water component is less than the high salinity brine. In various cases, the high salinity brine comprises water with a salt concentration (e.g., chloride, sulphate and sodium) higher than 50 parts per thousand. In some cases, the salt comprises sodium chloride.

In many embodiments, the brine of step (a) has reduced amounts of sodium, potassium, chloride, sulphate, magnesium, and/or calcium ions via chemical precipitation, electrochemical methods, ion selective membranes, reverse osmosis, and/or selective precipitation by pH. In some cases, the chemical precipitation comprises contacting the sodium, potassium, chloride, sulphate, magnesium, and/or calcium ions with lime (calcium hydroxide, Ca(OH)₂) or alum (aluminum sulfate, Al₂(SO₄)₃) which forms insoluble precipitates with the sodium, potassium, chloride, sulphate, magnesium, and/or calcium ions. In various cases, the chemical precipitation occurs before step (a) and/or during step (a).

In various embodiments, the reduced salinity water component is added to a natural or artificial body of water. In some cases, the reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In numerous embodiments, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process (as illustrated in FIG. 5) can more efficiently be performed, since, at least in part, because the sulphates and chlorides have already been removed. The extracted sodium may be isolated into a pure sodium product.

In additional embodiments, the method permits production by a desalination facility of a brine having a higher salt concentration relative to a method where the desalination facility produces brine as wastewater to be added to a natural or artificial body of water. In some cases, the relative salt concentration is up to 5-fold higher, e.g., about 1-fold, 2-fold, 3-fold, 4-fold, or about 5-fold and any range or value therebetween.

In several embodiments, the method permits production of more fresh water by a desalination facility relative to a method where the desalination facility produces brine as wastewater to be added to a natural or artificial body of water. In some cases, the amount of fresh water produced is up to 5-fold higher, e.g., about 1-fold, 2-fold, 3-fold, 4-fold, or about 5-fold and any range or value therebetween.

In embodiments, the steps (a) and/or (b) occurs in a location within the desalination facility, adjacent to the desalination facility, or downstream from the desalination facility. In some cases, step (a) and/or step (b) occurs in a pipeline within the desalination facility, adjacent to the desalination facility, or downstream from the desalination facility. In certain cases, the pressure in step (b) is applied to the pipeline utilizing a series of pipe reducers which creates pressure sufficient to make the conversion process (e.g., chemical reactions) more efficient. In various cases, a pipe reducer is a mechanical device used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

In some embodiments, the steps (a) and/or (b) occurs in a location distant from the desalination facility. In some cases, step (a) and/or step (b) occurs in a pipeline within the desalination facility, adjacent to the desalination facility, or downstream from the desalination facility. In certain cases, the pressure in step (b) is applied to the pipeline utilizing a series of pipe reducers.

In various cases, a pipe reducer is a mechanical device used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

In embodiments, the pipeline is between about 12 inches and about 18 inches in diameter. In some cases, the diameter of the pipeline is greater than 18 inches, e.g., a diameter capable of discharging a million gallons (or more) per day.

In many embodiments, the steps (a) and/or (b) occurs in a location within the desalination facility, adjacent to the desalination facility, or downstream from the desalination facility and steps (a) and/or (b) occurs in a tank or basin.

In various embodiments, the steps (a) and/or (b) occurs in a location distant from the desalination facility and steps (a) and/or (b) occurs in a tank or basin.

In numerous embodiments, the pozzolan comprises silica (SiO₂), alumina (Al₂O₃), and/or iron oxide (Fe₂O₃).

In embodiments, the pozzolan is a natural pozzolan or a man-made pozzolan.

In additional embodiments, the pozzolan comprises or is obtained from one or more of volcanic rock (e.g., rhyolite, olivine, obsidian, pitchstone, pumice, basalt or trap, or andesite); volcanic ash; sedimentary clays or shales' calcined clays; rice husk ash; diatomaceous earth; metakaolin; and olivine.

In several embodiments, use of pozzolan avoids the problem of sourcing cementitious material that is used in standard concrete manufacturing. In some cases, avoiding the problem of sourcing cementitious material further reduces the amount of energy required and/or waste CO₂ produced relative to standard concrete manufacturing. Without wishing to be bound by theory, while standard cement manufacturing is responsible for about 10% of the world's CO₂ emissions.

In embodiments, the cementitious aggregate composition absorbs more carbon dioxide during its manufacture than is emitted.

In some embodiments, the cementitious aggregate composition absorbs and/or retains at least 0.04 kg CO₂ per kg of composition.

In any embodiment, the CO₂ is chemically reacted to form a crystalline form of carbon.

In various embodiments, the CO₂ is absorbed into the aggregate cementitious as a crystalline form of carbon. Thus, the CO₂ is permanently removed from the atmosphere.

In many embodiments, the CO₂ of step (a) is from industrial waste, from environmental sources, or from molecular capture. In some cases, the industrial waste relates to alcoholic fermentation or the burning of fossil fuels.

In various embodiments, the CO₂ of step (a) is provided in gaseous, solid, and/or as a supercritical fluid form or as a dissolved gas.

In numerous embodiments, the CO₂ of step (a) is incorporated into the cementitious aggregate composition as a carbonate.

In additional embodiments, step (a) further comprises adding an activator. In various cases, the activator may be any sodium, lithium or potassium salt or hydroxide and any alkali or alkali earth metal ions. In some cases, the activator is Mg(OH)₂, MgO, Ca(OH)₂, CaCO₃, Al₂(SO₄)₃, and/or CaO. In some cases, the activator, e.g., Mg(OH)₂, MgO, Ca(OH)₂ and/or CaO, are not obtained by a pyroclastic process.

In various tank precipitation methods, an activator is preferably included in the combination.

In combinations comprising latently hydraulic material, an activator is preferably included in the combination.

In several embodiments, the cementitious aggregate composition forms particles of various shapes, sizes, and textures. In some cases, the particles have average sizes ranging from 0.0625 mm to about 40.0 mm in diameter. In various cases, the particles have generally rounded or oval shapes. The particles may comprise pores. In certain cases, the particles comprise more numerous pores per unit volume and/or larger average pores than a standard cement. In some cases, a standard cement has pores of 1.5 to 2.0 nm. The particles may be less dense per unit volume than a standard cement.

In embodiments, the particle has a diameter of about 0.0625 mm to about 40.0 mm. In embodiments the particle has a diameter of 0.0625 mm, 0.075 mm, 0.0825 mm, 0.0900 mm, 0.125 mm, 0.187 mm, 0.250 mm, 0.312 mm, 0.375 mm, 0.437 mm, 0.500 mm, 0.562 mm, 0.625 mm, 0.687 mm, 0.750 mm, 0.812 mm, 0.875, 0.937 mm, 1.00 mm, 1.0625 mm, 1.075 mm, 1.0825 mm, 1.0900 mm, 1.125 mm, 1.187 mm, 1.250 mm, 1.312 mm, 1.375 mm, 1.437 mm, 1.500 mm, 1.562 mm, 1.625 mm, 1.687 mm, 1.750 mm, 1.812 mm, 1.875, 1.937 mm, 2.00 mm, 2.0625 mm, 2.075 mm, 2.0825 mm, 2.0900 mm, 2.125 mm, 2.187 mm, 2.250 mm, 2.312 mm, 2.375 mm, 2.437 mm, 2.500 mm, 2.562 mm, 2.625 mm, 2.687 mm, 2.750 mm, 2.812 mm, 2.875, 2.937 mm, 3.00 mm, 3.0625 mm, 3.075 mm, 3.0825 mm, 3.0900 mm, 3.125 mm, 3.187 mm, 3.250 mm, 3.312 mm, 3.375 mm, 3.437 mm, 3.500 mm, 3.562 mm, 3.625 mm, 3.687 mm, 3.750 mm, 3.812 mm, 3.875, 3.937 mm, 4.00 mm, 4.0625 mm, 4.075 mm, 4.0825 mm, 4.0900 mm, 4.125 mm, 4.187 mm, 4.250 mm, 4.312 mm, 4.375 mm, 4.437 mm, 4.500 mm, 4.562 mm, 4.625 mm, 4.687 mm, 4.750 mm, 4.812 mm, 4.875, 4.937 mm, 5.00 mm, 5.0625 mm, 5.075 mm, 5.0825 mm, 5.0900 mm, 5.125 mm, 5.187 mm, 5.250 mm, 5.312 mm, 5.375 mm, 5.437 mm, 5.500 mm, 5.562 mm, 5.625 mm, 5.687 mm, 5.750 mm, 5.812 mm, 5.875, 5.937 mm, 6.00 mm, 6.50 mm, 7.00 mm, 7.50 mm, 8.00 mm, 8.50 mm, 9.00 mm, 9.50 mm, 10.0 mm, 10.5 mm, 11.0 mm, 11.5 mm, 12.0 mm, 12.5 mm, 13.0 mm, 13.5 mm, 14.0 mm, 14.5, mm, 15.0 mm, 15.5 mm, 16.0 mm, 16.5 mm, 17.0 mm, 17.5 mm, 18.0 mm, 18.5 mm, 19.0 mm, 19.5 mm, 20.0 mm, 21.5 mm, 22.0 mm, 22.5 mm, 23.0 mm, 23.5 mm, 24.0 mm, 24.5, mm, 25.0 mm, 25.5 mm, 26.0 mm, 26.5 mm, 27.0 mm, 27.5 mm, 28.0 mm, 28.5 mm, 29.0 mm, 29.5 mm, 30.0 mm, 30.5 mm, 31.0 mm, 31.5 mm, 32.0 mm, 32.5 mm, 33.0 mm, 33.5 mm, 34.0 mm, 34.5, mm, 35.0 mm, 35.5 mm, 36.0 mm, 36.5 mm, 37.0 mm, 37.5 mm, 38.0 mm, 38.5 mm, 39.0 mm, 39.5 mm, 40.0 mm; or values between the foregoing ranges including endpoints.

In embodiments, the cementitious aggregate composition comprises more CO₂ per gram than a standard cement. In some cases, the cementitious aggregate composition comprises up to 50% more CO₂ per gram than a standard cement.

In some embodiments, the cementitious aggregate composition comprises more calcium, silicon, aluminum, magnesium and/or iron per gram than a standard cement. In some cases, the cementitious aggregate composition comprises up to 50% more calcium, silicon, aluminum, magnesium and/or iron per gram than a standard cement.

In numerous embodiments, the density of the cementitious aggregate composition is from about 1000 g/cc to about 5000 g/cc, e.g., about 1000 g/cc, 1500 g/cc, 2000 g/cc, 2500 g/cc, 3000 g/cc, 3500 g/cc, 4000 g/cc, 4500 g/cc, or about 5000 g/cc. In some case the density is about 2500 g/cc.

In many embodiments, the standard cement comprises CaO, CaCO₃, SiO₂, Al₂O₃, Fe₂O₃, and CaSO₄·H₂O.

In various embodiments, the standard cement comprises a slaked or hydraulic dolomitic or calcareous lime blended with a natural or man-made pozzolanic or latently hydraulic material.

In numerous embodiments, the standard cement, e.g., a standard hydrated cement, is a Portland Cement comprising cement clinker rather than pozzolan.

In additional embodiments, the cementitious aggregate composition is deposited into a natural or artificial body of water. In some cases, when deposited into the natural or artificial body of water, the cementitious aggregate composition remains where deposited or when deposited into the natural or artificial body of water, a portion of the cementitious aggregate composition is distributed away from the deposit site. In various cases, the cementitious aggregate composition is distributed away from the deposit site by water movement or currents, erosion, or animal activity. In numerous cases, the cementitious aggregate composition permits attachment by flora, e.g., algae, and/or fauna, e.g., coral. In many cases, the cementitious aggregate composition permits a preferred level of colonization by flora and/or fauna relative to a standard cement and/or the cementitious aggregate composition permits more plentiful attachment by flora and/or fauna relative to a standard cement.

In embodiments, the cementitious aggregate composition comprises any sodium, lithium or Potassium salt or hydroxide and any alkali or alkali earth metal ions and including Mg(OH)₂, MgO, Ca(OH)₂, CaCO₃, Al₂(SO₄)₃, CaO, CaSO₄·H₂O, SiO₂, Fe₂O₃, Al₂O₃ and/or Brucite.

In several embodiments, the cementitious aggregate composition comprises MgO. In some cases, the cementitious aggregate composition does not substantially comprise MgO obtained from a calcination reaction.

In some embodiments, step (b) produces exothermic heat. In some cases, the exothermic heat is transformable from thermal energy into electrical energy.

In many embodiments, the pressure in step (b) is from about ambient (1 bar) to about 250 bar.

In various embodiments, the pressure in step (b) is from about 100 bar to about 250 bar. As examples, the pressure in about 101 bar, 102 bar, 103 bar, 104 bar, 105 bar, 106 bar, 107 bar, 108 bar, 109 bar, 110 bar, 111 bar, 112 bar, 113 bar, 114 bar, 115 bar, 116 bar, 117 bar, 118 bar, 119 bar, 120 bar, 121 bar, 122 bar, 123 bar, 124 bar, 125 bar, 126 bar, 127 bar, 128 bar, 129 bar, 130 bar, 131 bar, 132 bar, 133 bar, 134 bar, 135 bar, 136 bar, 137 bar, 138 bar, 139 bar, 140 bar, 141 bar, 142 bar, 143 bar, 144 bar, 145 bar, 146 bar, 147 bar, 148 bar, 149 bar, 150 bar, 151 bar, 152 bar, 153 bar, 154 bar, 155 bar, 156 bar, 157 bar, 158 bar, 159 bar, 160 bar, 161 bar, 162 bar, 163 bar, 164 bar, 165 bar, 166 bar, 167 bar, 168 bar, 169 bar, 170 bar, 171 bar, 172 bar, 173 bar, 174 bar, 175 bar, 176 bar, 177 bar, 178 bar, 179 bar, 180 bar, 181 bar, 182 bar, 183 bar, 184 bar, 185 bar, 186 bar, 187 bar, 188 bar, 189 bar, 190 bar, 191 bar, 192 bar, 193 bar, 194 bar, 195 bar, 196 bar, 197 bar, 198 bar, 199 bar, 200 bar, 101 bar, 202 bar, 203 bar, 204 bar, 205 bar, 206 bar, 207 bar, 208 bar, 209 bar, 210 bar, 211 bar, 212 bar, 213 bar, 214 bar, 215 bar, 216 bar, 217 bar, 218 bar, 219 bar, 220 bar, 221 bar, 222 bar, 223 bar, 224 bar, 225 bar, 226 bar, 227 bar, 228 bar, 229 bar, 230 bar, 231 bar, 232 bar, 233 bar, 234 bar, 235 bar, 236 bar, 237 bar, 238 bar, 239 bar, 240 bar, 241 bar, 242 bar, 243 bar, 244 bar, 245 bar, 246 bar, 247 bar, 248 bar, 249 bar, or about 250 bar, and any range or value therebetween.

In numerous embodiments, the pressure in step (b) is from about ambient (1 bar) to about 100 bar. As examples, the pressure is about 1 bar, 2 bar, 3 bar, 4 bar, 5 bar, 6 bar, 7 bar, 8 bar, 9 bar, 10 bar, 11 bar, 12 bar, 13 bar, 14 bar, 15 bar, 16 bar, 17 bar, 18 bar, 19 bar, 20 bar, 21 bar, 22 bar, 23 bar, 24 bar, 25 bar, 26 bar, 27 bar, 28 bar, 29 bar, 30 bar, 31 bar, 32 bar, 33 bar, 34 bar, 35 bar, 36 bar, 37 bar, 38 bar, 39 bar, 40 bar, 41 bar, 42 bar, 43 bar, 44 bar, 45 bar, 46 bar, 47 bar, 48 bar, 49 bar, 50 bar, 51 bar, 52 bar, 53 bar, 54 bar, 55 bar, 56 bar, 57 bar, 58 bar, 59 bar, 60 bar, 61 bar, 62 bar, 63 bar, 64 bar, 65 bar, 66 bar, 67 bar, 68 bar, 69 bar, 70 bar, 71 bar, 72 bar, 73 bar, 74 bar, 75 bar, 76 bar, 77 bar, 78 bar, 79 bar, 80 bar, 81 bar, 82 bar, 83 bar, 84 bar, 85 bar, 86 bar, 87 bar, 88 bar, 89 bar, 90 bar, 91 bar, 92 bar, 93 bar, 94 bar, 95 bar, 96 bar, 97 bar, 98 bar, 99 bar, or about 100 bar, and any range or value therebetween.

In various embodiments, the pressure in step (b) is greater than about 250 bar. For example, the pressure in step (b) is greater than, 250 bar, 500 bar, 750 bar, 1000 bar, 1250 bar, 1500 bar, 1750 bar, 2000 bar, 2250 bar, 2500 bar, 2750 bar, 3000 bar, 3250 bar, 3500 bar, 3750 bar, 4000 bar, 4250 bar, 4500 bar, 4750 bar, 5000 bar and any range or value therebetween. In various cases the pressure in step (b) is greater than 250 bar, 260 bar, 270 bar, 280 bar, 290 bar, 300 bar, 310 bar, 320 bar, 330 bar, 340 bar, 350 bar, 360 bar, 370 bar, 380 bar, 390 bar, 400 bar, 410 bar, 420 bar, 430 bar, 440 bar, 450 bar, 460 bar, 470 bar, 480 bar, 490 bar, 500 bar and any range or value therebetween. The pressure in step (b) may be greater than 500 bar, 510 bar, 520 bar, 530 bar, 540 bar, 550 bar, 560 bar, 570 bar, 580 bar, 590 bar, 600 bar, 610 bar, 620 bar, 630 bar, 640 bar, 650 bar, 660 bar, 670 bar, 680 bar, 690 bar, 700 bar, 710 bar, 720 bar, 730 bar, 740 bar, 750 bar, 760 bar, 770 bar, 780 bar, 790 bar, 800 bar, 810 bar, 820 bar, 830 bar, 840 bar, 850 bar, 860 bar, 870 bar, 880 bar, 890 bar, 900 bar, 910 bar, 920 bar, 930 bar, 940 bar, 950 bar, 960 bar, 970 bar, 980 bar, 990 bar, 1000 bar and any range or value therebetween.

In additional embodiments, the temperature of step (a) and/or step (b) is from about 1° C. to about 100° C. As examples, the temperature is about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or about 100° C., and any range or value therebetween. In various cases, heat is provided to achieve a temperature of greater than 100° C. and up to 3000° C.

In any embodiment, heat is applied to the mixture and/or the mixture is heated via an additional. The applied heat increases the rate of reaction and/or the extent of reaction and/or activates the latent hydraulic nature of some components. In several embodiments, the duration of step (b) is from about 1 minute to about 10 hours. As examples the duration is about 10 minutes, 20 minutes, 30 minutes, 40 minutes, 50 minutes, 60 minutes, 70 minutes, 80 minutes, 90 minutes, 2 hours, 3 hours, 4 hours, 5 hours, 6 hours, 7 hours, 8 hours, 9 hours, or about 10 hours, and any range or value therebetween.

In several embodiments, the duration of step (b) is from about 10 hours to about 10 days. As examples the duration is about 10 hours, 20 hours, 30 hours, 40 hours, 50 hours, 60 hours, 70 hours, 80 hours, 90 hours, 2 days, 3 days, 4 days, 5 days, 6 days, 7 days, 8 days, 9 days, or about 10 days, and any range or value therebetween.

In embodiments, the duration of step (b) is from about 1 minute to about 10 minutes. As examples the duration is about 1 minute, 2 minutes, 3 minutes, 4 minutes, 5 minutes, 6 minutes, 7 minutes, 8 minutes, 9 minutes, or about 10 minutes, and any range or value therebetween. In numerous embodiments, the ratio (weight to weight or weight to volume or volume to volume) of the high salinity brine to pozzolan and/or a latently hydraulic material varies from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the high salinity brine to CO₂ varies from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In some embodiments, the ratio (weight to weight or weight to volume or volume to volume) of the pozzolan and/or a latently hydraulic material to CO₂ varies from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In some embodiments, a filler material or another additive is added to the combination of step (a). In some cases, a filler material or another additive comprises sand, gravel, lightweight aggregate, or crushed stone, and a combination thereof.

In embodiments, the ratio (weight to weight or weight to volume or volume to volume) of the aggregate to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In various embodiments, the ratio (weight to weight or weight to volume or volume to volume) of the aggregate to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In many embodiments, the latently hydraulic material is a slag. In some cases, the slag is added in step (a) in addition to the added pozzolan. In alternate cases, the slag is added in step (a) instead of pozzolan. In some cases, the slag is added in step (a) instead of an activator.

In embodiments, the method further comprises isolating the cementitious aggregate composition from the reduced salinity water component or other liquid components. In some cases, the isolating comprises straining to capture the cementitious aggregate composition.

In various embodiments, the cementitious aggregate composition is molded into a shaped cementitious composition. Without wishing to be bound by theory, particles of the cementitious aggregate composition can stick to each other, thereby permitting the molding of the composition into shapes. In some case, molding may not require an additional binder to keep the molded shape and in other cases, molding may require an additional binder to keep the molded shape. In some cases, the shaped cementitious composition permits attachment by flora, e.g., algae, and/or fauna, e.g., coral, relative to a standard cement. In various cases, the shaped cementitious composition permits stronger attachment by flora and/or fauna relative to a standard cement and/or the shaped cementitious composition permits more plentiful attachment by flora and/or fauna relative to a standard cement. When molded, the shaped cementitious composition comprises gaps between particles of the aggregate such that the of the shaped cementitious composition is has a more porous surface, is more porous throughout its volume, and is less dense than a standard cement. The gaps of a shaped cementitious composition may be up to 500 nm in diameter. Additionally, a shaped cementitious composition may have up to 85% of the volume being void (e.g., the sum of all gaps) whereas a standard cement may have about 40% of the volume being void.

The cementitious aggregate composition be used in any way that another cementitious aggregates can be used.

The shape of the shaped cementitious composition may be any shape capable of providing a substrate for aquatic flora and/or fauna attachment, as examples. The shape could be planar, circular, rounded, elongated, flat, rectangular, or any combination thereof. In some embodiments, the shape is pyramidal with closed surfaces. In other embodiments, the shape is pyramidal with open surfaces such that the shape comprises four bars or cylinders that form a square base and four bars or cylinders each originating at a corner of the square and converging to form the pyramid's apex.

The shaped cementitious aggregate composition be used in any way that any cement can be used.

In embodiments, the cementitious composition further comprises at least one filler material or other additive, the at least one filler or other additive can be pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

In some embodiments, the ratio (weight to weight or weight to volume or volume to volume) of the filler material or other additive to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In various embodiments, the ratio (weight to weight or weight to volume or volume to volume) of the filler material or other additive to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

A further aspect of the present disclosure is a method for providing substrate for aquatic flora and/or fauna attachment, the method comprising depositing a herein-disclosed cementitious aggregate composition into a natural or artificial body of water which comprises aquatic flora and/or fauna in need of an attachment.

In some cases, when deposited into the natural or artificial body of water, the cementitious aggregate composition remains where deposited or when deposited into the natural or artificial body of water, a portion of the cementitious aggregate composition is distributed away from the deposit site. In various cases, the cementitious aggregate composition is distributed away from the deposit site by water movement or currents, erosion, or animal activity. In numerous cases, the cementitious aggregate composition permits attachment by flora, e.g., algae, and/or fauna, e.g., coral. In many cases, the cementitious aggregate composition permits a preferred level of colonization by flora and/or fauna relative to a standard cement and/or the cementitious aggregate composition permits more plentiful attachment by flora and/or fauna relative to a standard cement.

In an aspect, the present disclosure provides a method for providing substrate for aquatic flora and/or fauna attachment, the method comprising depositing any herein-disclosed shaped cementitious composition into a natural or artificial body of water which comprises aquatic flora and/or fauna in need of an attachment. In this aspect, the cementitious aggregate composition is molded into a shaped cementitious composition.

In embodiments, the shaped cementitious composition permits attachment by flora, e.g., algae, and/or fauna, e.g., coral, relative to a standard cement. In various cases, the shaped cementitious composition permits stronger attachment by flora and/or fauna relative to a standard cement and/or the shaped cementitious composition permits more plentiful attachment by flora and/or fauna relative to a standard cement.

In some embodiments, a filler material or another additive is added to the combination of step (a). In some cases, the filler material or the other additive comprises sand, gravel, lightweight aggregate, or crushed stone, and a combination thereof.

In numerous embodiments, the ratio (weight to weight or weight to volume or volume to volume) of the aggregate to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the aggregate to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In many embodiments, the latently hydraulic material is a slag. In some cases, the slag is added in step (a) in addition to the added pozzolan. In alternate cases, the slag is added in step (a) instead of pozzolan. In some cases, the slag is added in step (a) instead of an activator.

In embodiments, the method further comprises isolating the cementitious aggregate composition from the reduced salinity water component or other liquid components. In some cases, the isolating comprises straining to capture the cementitious aggregate composition.

In various embodiments, the cementitious aggregate composition is molded into a shaped cementitious composition. Without wishing to be bound by theory, particles of the cementitious aggregate composition can stick to each other, thereby permitting the molding of the composition into shapes. In some case, molding may not require an additional binder to keep the molded shape and in other cases, molding may require an additional binder to keep the molded shape. In some cases, the shaped cementitious composition permits attachment by flora, e.g., algae, and/or fauna, e.g., coral, relative to a standard cement. In various cases, the shaped cementitious composition permits stronger attachment by flora and/or fauna relative to a standard cement and/or the shaped cementitious composition permits more plentiful attachment by flora and/or fauna relative to a standard cement. When molded, the shaped cementitious composition comprises gaps between particles of the aggregate such that the of the shaped cementitious composition is has a more porous surface, is more porous throughout its volume, and is less dense than a standard cement. The gaps of a shaped cementitious composition may be up to 500 nm in diameter. Additionally, a shaped cementitious composition may have up to 85% of the volume being void (e.g., the sum of all gaps) whereas a standard cement may have about 40% of the volume being void.

The shape of the shaped cementitious composition may be any shape capable of providing a substrate for aquatic flora and/or fauna attachment. The shape could be planar, circular, rounded, elongated, flat, rectangular, or any combination thereof. In some embodiments, the shape is pyramidal with closed surfaces. In other embodiments, the shape is pyramidal with open surfaces such that the shape comprises four bars or cylinders that form a square base and four bars or cylinders each originating at a corner of the square and converging to form the pyramid's apex.

In many embodiments, the cementitious composition further comprises at least one filler material or other additive, the at least one filler or other additive can be pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

In some embodiments, the ratio (weight to weight or weight to volume or volume to volume) of the filler material or other additive to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the filler material or other additive to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

The methods, steps, and/or features thereof disclosed in FIG. 1 or FIG. 3 to FIG. 5 may be incorporated into any of the herein-disclosed aspect or embodiment.

In various embodiments, a mixture which is transformed into a cementitious aggregate composition and a reduced salinity water component, may comprise one of the following combinations:
(1) brine, (2) CO₂, and (3) a natural pozzolan;
(1) brine, (2) CO₂, (3) a natural pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a man-made pozzolan;
(1) brine, (2) CO₂, (3) a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a latently hydraulic material;
(1) brine, (2) CO₂, (3) a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan and a man-made pozzolan;
(1) brine, (2) CO₂, (3) a natural pozzolan and a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan and a latently hydraulic material;
(1) brine, (2) CO₂, (3) a natural pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a man-made pozzolan and a latently hydraulic material;
(1) brine, (2) CO₂, (3) a man-made pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan, a man-made pozzolan, and a latently hydraulic material; or
(1) brine, (2) CO₂, (3) a natural pozzolan, a man-made pozzolan, and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO).

In various tank precipitation methods, an activator is preferably included in the combination. In combinations comprising latently hydraulic material, an activator is preferably included in the combination.

Any method or method step disclosed herein is applicable to any other herein-disclosed method or method step. In other words, any aspect or embodiment described herein can be combined with any other aspect or embodiment as disclosed herein.

### Compositions

Another aspect of the present disclosure is a cementitious aggregate composition obtained by any herein-disclosed method.

In embodiments, the cementitious aggregate composition is obtained by a method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a reduced salinity water component and a cementitious aggregate composition.

In several embodiments, the high salinity brine, CO₂, and pozzolan and/or a latently hydraulic material are combined simultaneously.

In some embodiments, the high salinity brine, CO₂, and pozzolan and/or a latently hydraulic material are combined sequentially with the high salinity brine and CO₂ combined first.

In various embodiments, the high salinity brine, CO₂, and pozzolan and/or a latently hydraulic material are combined sequentially with the CO₂ and the pozzolan combined first.

In numerous embodiments, the high salinity brine, CO₂, and pozzolan and/or a latently hydraulic material are combined sequentially with the high salinity brine and the pozzolan combined first.

In additional embodiments, the high salinity brine is obtained from a desalination facility, is natural seawater, or an industrial brine.

In several embodiments, the salt concentration of the high salinity brine is greater than or about equal to the salt concentration of seawater. In some cases, the salt comprises sodium chloride.

In embodiments, the salt concentration of the reduced salinity water component is less than or about equal to the salt concentration of seawater. In some cases, the salt comprises sodium chloride.

In some embodiments, the salt concentration of the reduced salinity water component is less than the high salinity brine. In various cases, the high salinity brine comprises water with a salt concentration (e.g., chloride, sulphate and sodium) higher than 50 parts per thousand. In some cases, the salt comprises sodium chloride.

In many embodiments, the brine of step (a) has reduced amounts of sodium, potassium, chloride, sulphate, magnesium, and/or calcium ions via chemical precipitation, electrochemical methods, ion selective membranes, reverse osmosis, and/or selective precipitation by pH. In some cases, the chemical precipitation comprises contacting the sodium, potassium, chloride, sulphate, magnesium, and/or calcium ions with lime (calcium hydroxide, Ca(OH)₂) or alum (aluminum sulfate, Al₂(SO₄)₃) which forms insoluble precipitates with the sodium, potassium, chloride, sulphate, magnesium, and/or calcium ions. In various cases, the chemical precipitation occurs before step (a) and/or during step (a).

In various embodiments, the reduced salinity water component is added to a natural or artificial body of water. In some cases, the reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In numerous embodiments, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process can more efficiently be performed, since, at least in part, because the sulphates and chlorides have already been removed. The extracted sodium may be isolated into a pure sodium product.

In additional embodiments, the method permits production by a desalination facility of a brine having a higher salt concentration relative to a method where the desalination facility produces brine as wastewater to be added to a natural or artificial body of water. In some cases, the relative salt concentration is up to 5-fold higher, e.g., about 1-fold, 2-fold, 3-fold, 4-fold, or about 5-fold and any range or value therebetween.

In several embodiments, the method permits production of more fresh water by a desalination facility relative to a method where the desalination facility produces brine as wastewater to be added to a natural or artificial body of water. In some cases, the amount of fresh water produced is up to 5-fold higher, e.g., about 1-fold, 2-fold, 3-fold, 4-fold, or about 5-fold and any range or value therebetween.

In embodiments, the steps (a) and/or (b) occurs in a location within the desalination facility, adjacent to the desalination facility, or downstream from the desalination facility. In some cases, step (a) and/or step (b) occurs in a pipeline within the desalination facility, adjacent to the desalination facility, or downstream from the desalination facility. In certain cases, the pressure in step (b) is applied to the pipeline utilizing a series of pipe reducers. In various cases, a pipe reducer is a mechanical device used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

In some embodiments, the steps (a) and/or (b) occurs in a location distant from the desalination facility. In some cases, step (a) and/or step (b) occurs in a pipeline within the desalination facility, adjacent to the desalination facility, or downstream from the desalination facility. In certain cases, the pressure in step (b) is applied to the pipeline utilizing a series of pipe reducers. In various cases, a pipe reducer is a mechanical device used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

In embodiments, the pipeline is between about 12 inches and about 18 inches in diameter.

In many embodiments, the steps (a) and/or (b) occurs in a location within the desalination facility, adjacent to the desalination facility, or downstream from the desalination facility and steps (a) and/or (b) occurs in a tank or basin.

In various embodiments, the steps (a) and/or (b) occurs in a location distant from the desalination facility and steps (a) and/or (b) occurs in a tank or basin.

In numerous embodiments, the pozzolan comprises silica (SiO₂), alumina (Al₂O₃), and/or iron oxide (Fe₂O₃).

In embodiments, the pozzolan is a natural pozzolan or a man-made pozzolan.

In additional embodiments, the pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

In several embodiments, use of pozzolan avoids the problem of sourcing cementitious material that is used in standard concrete manufacturing. In some cases, avoiding the problem of sourcing cementitious material further reduces the amount of energy required and/or waste CO₂ produced relative to standard concrete manufacturing. Without wishing to be bound by theory, while standard cement manufacturing is responsible for about 10% of the world's CO₂ emissions.

In embodiments, the cementitious aggregate composition absorbs more carbon dioxide during its manufacture than is emitted.

In some embodiments, the cementitious aggregate composition absorbs and/or retains at least 0.04 kg CO₂ per kg of composition.

In any embodiment, the CO₂ is chemically reacted to form a crystalline form of carbon.

In various embodiments, the CO₂ is absorbed into the aggregate cementitious as a crystalline form of carbon. Thus, the CO₂ is permanently removed from the atmosphere. In many embodiments, the CO₂ of step (a) is from industrial waste, from environmental sources, or from molecular capture. In some cases, the industrial waste relates to alcoholic fermentation or the burning of fossil fuels.

In various embodiments, the CO₂ of step (a) is provided in gaseous, solid, and/or as a supercritical fluid form or as a dissolved gas.

In numerous embodiments, the CO₂ of step (a) is incorporated into the cementitious aggregate composition as a carbonate. Without wishing to be bound by theory, when CO₂ reacts with calcium hydroxide (Ca(OH)₂), which is commonly present in cementitious systems, it can form calcium carbonate (CaCO₃). This reaction contributes to the hardening of the cementitious aggregate composition, making it more durable and cementitious in nature.

In additional embodiments, step (a) further comprises adding an activator. In various cases, the activator may be any sodium, lithium or potassium salt or hydroxide and any alkali or alkali earth metal ions. In some cases, the activator is Mg(OH)₂, MgO, Ca(OH)₂, CaCO₃, Al₂(SO₄)₃, and/or CaO.

In various tank precipitation methods, an activator is preferably included in the combination.

In combinations comprising latently hydraulic material, an activator is preferably included in the combination.

In several embodiments, the cementitious aggregate composition forms particles of various shapes, sizes, and textures. In some cases, the particles have average sizes ranging from 0.0625 mm to about 40.0 mm in diameter. In various cases, the particles have generally rounded or oval shapes. The particles may comprise pores. In certain cases, the particles comprise more numerous pores per unit volume and/or larger average pores than a standard cement. The particles may be less dense per unit volume than a standard cement.

In embodiments, the particle has a diameter of about 0.0625 mm to about 40.0 mm. In embodiments the particle has a diameter of 0.0625 mm, 0.075 mm, 0.0825 mm, 0.0900 mm, 0.125 mm, 0.187 mm, 0.250 mm, 0.312 mm, 0.375 mm, 0.437 mm, 0.500 mm, 0.562 mm, 0.625 mm, 0.687 mm, 0.750 mm, 0.812 mm, 0.875, 0.937 mm, 1.00 mm, 1.0625 mm, 1.075 mm, 1.0825 mm, 1.0900 mm, 1.125 mm, 1.187 mm, 1.250 mm, 1.312 mm, 1.375 mm, 1.437 mm, 1.500 mm, 1.562 mm, 1.625 mm, 1.687 mm, 1.750 mm, 1.812 mm, 1.875, 1.937 mm, 2.00 mm, 2.0625 mm, 2.075 mm, 2.0825 mm, 2.0900 mm, 2.125 mm, 2.187 mm, 2.250 mm, 2.312 mm, 2.375 mm, 2.437 mm, 2.500 mm, 2.562 mm, 2.625 mm, 2.687 mm, 2.750 mm, 2.812 mm, 2.875, 2.937 mm, 3.00 mm, 3.0625 mm, 3.075 mm, 3.0825 mm, 3.0900 mm, 3.125 mm, 3.187 mm, 3.250 mm, 3.312 mm, 3.375 mm, 3.437 mm, 3.500 mm, 3.562 mm, 3.625 mm, 3.687 mm, 3.750 mm, 3.812 mm, 3.875, 3.937 mm, 4.00 mm, 4.0625 mm, 4.075 mm, 4.0825 mm, 4.0900 mm, 4.125 mm, 4.187 mm, 4.250 mm, 4.312 mm, 4.375 mm, 4.437 mm, 4.500 mm, 4.562 mm, 4.625 mm, 4.687 mm, 4.750 mm, 4.812 mm, 4.875, 4.937 mm, 5.00 mm, 5.0625 mm, 5.075 mm, 5.0825 mm, 5.0900 mm, 5.125 mm, 5.187 mm, 5.250 mm, 5.312 mm, 5.375 mm, 5.437 mm, 5.500 mm, 5.562 mm, 5.625 mm, 5.687 mm, 5.750 mm, 5.812 mm, 5.875, 5.937 mm, 6.00 mm, 6.50 mm, 7.00 mm, 7.50 mm, 8.00 mm, 8.50 mm, 9.00 mm, 9.50 mm, 10.0 mm, 10.5 mm, 11.0 mm, 11.5 mm, 12.0 mm, 12.5 mm, 13.0 mm, 13.5 mm, 14.0 mm, 14.5, mm, 15.0 mm, 15.5 mm, 16.0 mm, 16.5 mm, 17.0 mm, 17.5 mm, 18.0 mm, 18.5 mm, 19.0 mm, 19.5 mm, 20.0 mm, 21.5 mm, 22.0 mm, 22.5 mm, 23.0 mm, 23.5 mm, 24.0 mm, 24.5, mm, 25.0 mm, 25.5 mm, 26.0 mm, 26.5 mm, 27.0 mm, 27.5 mm, 28.0 mm, 28.5 mm, 29.0 mm, 29.5 mm, 30.0 mm, 30.5 mm, 31.0 mm, 31.5 mm, 32.0 mm, 32.5 mm, 33.0 mm, 33.5 mm, 34.0 mm, 34.5, mm, 35.0 mm, 35.5 mm, 36.0 mm, 36.5 mm, 37.0 mm, 37.5 mm, 38.0 mm, 38.5 mm, 39.0 mm, 39.5 mm, 40.0 mm; or values between the foregoing ranges including endpoints.

In embodiments, the cementitious aggregate composition comprises more CO₂ per gram than a standard cement. In some cases, the cementitious aggregate composition comprises up to 50% more CO₂ per gram than a standard cement.

In some embodiments, the cementitious aggregate composition comprises more calcium, silicon, aluminum, magnesium and/or iron per gram than a standard cement. In some cases, the cementitious aggregate composition comprises up to 50% more calcium, silicon, aluminum, magnesium and/or iron per gram than a standard cement.

In many embodiments, the standard cement comprises CaO, CaCO₃, SiO₂, Al₂O₃, Fe₂O₃, and CaSO₄·H₂O.

In various embodiments, the standard cement comprises a slaked or hydraulic dolomitic or calcareous lime blended with a natural or man-made pozzolanic or latently hydraulic material.

In numerous embodiments, the standard cement, e.g., a standard hydrated cement, is a Portland Cement comprising cement clinker rather than pozzolan.

In additional embodiments, the cementitious aggregate composition is deposited into a natural or artificial body of water. In some cases, when deposited into the natural or artificial body of water, the cementitious aggregate composition remains where deposited or when deposited into the natural or artificial body of water, a portion of the cementitious aggregate composition is distributed away from the deposit site. In various cases, the cementitious aggregate composition is distributed away from the deposit site by water movement or currents, erosion, or animal activity. In numerous cases, the cementitious aggregate composition permits attachment by flora, e.g., algae, and/or fauna, e.g., coral. In many cases, the cementitious aggregate composition permits a preferred level of colonization by flora and/or fauna relative to a standard cement and/or the cementitious aggregate composition permits more plentiful attachment by flora and/or fauna relative to a standard cement.

In embodiments, the cementitious aggregate composition comprises any sodium, lithium or Potassium salt or hydroxide and any alkali or alkali earth metal ions and including Mg(OH)₂, MgO, Ca(OH)₂, CaCO₃, Al₂(SO₄)₃, CaO, CaSO₄·H₂O, SiO₂, Fe₂O₃, Al₂O₃ and/or Brucite.

In several embodiments, the cementitious aggregate composition comprises MgO. In some cases, the cementitious aggregate composition does not substantially comprise MgO obtained from a calcination reaction.

The cementitious aggregate composition be used in any way that another cementitious aggregates can be used.

In some embodiments, step (b) produces exothermic heat. In some cases, the exothermic heat is transformable from thermal energy into electrical energy.

In many embodiments, the pressure in step (b) is from about ambient (1 bar) to about 250 bar.

In various embodiments, the pressure in step (b) is from about 100 bar to about 250 bar. As examples, the pressure in about 101 bar, 102 bar, 103 bar, 104 bar, 105 bar, 106 bar, 107 bar, 108 bar, 109 bar, 110 bar, 111 bar, 112 bar, 113 bar, 114 bar, 115 bar, 116 bar, 117 bar, 118 bar, 119 bar, 120 bar, 121 bar, 122 bar, 123 bar, 124 bar, 125 bar, 126 bar, 127 bar, 128 bar, 129 bar, 130 bar, 131 bar, 132 bar, 133 bar, 134 bar, 135 bar, 136 bar, 137 bar, 138 bar, 139 bar, 140 bar, 141 bar, 142 bar, 143 bar, 144 bar, 145 bar, 146 bar, 147 bar, 148 bar, 149 bar, 150 bar, 151 bar, 152 bar, 153 bar, 154 bar, 155 bar, 156 bar, 157 bar, 158 bar, 159 bar, 160 bar, 161 bar, 162 bar, 163 bar, 164 bar, 165 bar, 166 bar, 167 bar, 168 bar, 169 bar, 170 bar, 171 bar, 172 bar, 173 bar, 174 bar, 175 bar, 176 bar, 177 bar, 178 bar, 179 bar, 180 bar, 181 bar, 182 bar, 183 bar, 184 bar, 185 bar, 186 bar, 187 bar, 188 bar, 189 bar, 190 bar, 191 bar, 192 bar, 193 bar, 194 bar, 195 bar, 196 bar, 197 bar, 198 bar, 199 bar, 200 bar, 101 bar, 202 bar, 203 bar, 204 bar, 205 bar, 206 bar, 207 bar, 208 bar, 209 bar, 210 bar, 211 bar, 212 bar, 213 bar, 214 bar, 215 bar, 216 bar, 217 bar, 218 bar, 219 bar, 220 bar, 221 bar, 222 bar, 223 bar, 224 bar, 225 bar, 226 bar, 227 bar, 228 bar, 229 bar, 230 bar, 231 bar, 232 bar, 233 bar, 234 bar, 235 bar, 236 bar, 237 bar, 238 bar, 239 bar, 240 bar, 241 bar, 242 bar, 243 bar, 244 bar, 245 bar, 246 bar, 247 bar, 248 bar, 249 bar, or about 250 bar, and any range or value therebetween.

In numerous embodiments, the pressure in step (b) is from about ambient (1 bar) to about 100 bar. As examples, the pressure is about 1 bar, 2 bar, 3 bar, 4 bar, 5 bar, 6 bar, 7 bar, 8 bar, 9 bar, 10 bar, 11 bar, 12 bar, 13 bar, 14 bar, 15 bar, 16 bar, 17 bar, 18 bar, 19 bar, 20 bar, 21 bar, 22 bar, 23 bar, 24 bar, 25 bar, 26 bar, 27 bar, 28 bar, 29 bar, 30 bar, 31 bar, 32 bar, 33 bar, 34 bar, 35 bar, 36 bar, 37 bar, 38 bar, 39 bar, 40 bar, 41 bar, 42 bar, 43 bar, 44 bar, 45 bar, 46 bar, 47 bar, 48 bar, 49 bar, 50 bar, 51 bar, 52 bar, 53 bar, 54 bar, 55 bar, 56 bar, 57 bar, 58 bar, 59 bar, 60 bar, 61 bar, 62 bar, 63 bar, 64 bar, 65 bar, 66 bar, 67 bar, 68 bar, 69 bar, 70 bar, 71 bar, 72 bar, 73 bar, 74 bar, 75 bar, 76 bar, 77 bar, 78 bar, 79 bar, 80 bar, 81 bar, 82 bar, 83 bar, 84 bar, 85 bar, 86 bar, 87 bar, 88 bar, 89 bar, 90 bar, 91 bar, 92 bar, 93 bar, 94 bar, 95 bar, 96 bar, 97 bar, 98 bar, 99 bar, or about 100 bar, and any range or value therebetween.

In various embodiments, the pressure in step (b) is greater than about 250 bar. For example, the pressure in step (b) is greater than, 250 bar, 500 bar, 750 bar, 1000 bar, 1250 bar, 1500 bar, 1750 bar, 2000 bar, 2250 bar, 2500 bar, 2750 bar, 3000 bar, 3250 bar, 3500 bar, 3750 bar, 4000 bar, 4250 bar, 4500 bar, 4750 bar, 5000 bar and any range or value therebetween. In various cases the pressure in step (b) is greater than 250 bar, 260 bar, 270 bar, 280 bar, 290 bar, 300 bar, 310 bar, 320 bar, 330 bar, 340 bar, 350 bar, 360 bar, 370 bar, 380 bar, 390 bar, 400 bar, 410 bar, 420 bar, 430 bar, 440 bar, 450 bar, 460 bar, 470 bar, 480 bar, 490 bar, 500 bar and any range or value therebetween. The pressure in step (b) may be greater than 500 bar, 510 bar, 520 bar, 530 bar, 540 bar, 550 bar, 560 bar, 570 bar, 580 bar, 590 bar, 600 bar, 610 bar, 620 bar, 630 bar, 640 bar, 650 bar, 660 bar, 670 bar, 680 bar, 690 bar, 700 bar, 710 bar, 720 bar, 730 bar, 740 bar, 750 bar, 760 bar, 770 bar, 780 bar, 790 bar, 800 bar, 810 bar, 820 bar, 830 bar, 840 bar, 850 bar, 860 bar, 870 bar, 880 bar, 890 bar, 900 bar, 910 bar, 920 bar, 930 bar, 940 bar, 950 bar, 960 bar, 970 bar, 980 bar, 990 bar, 1000 bar and any range or value therebetween.

In additional embodiments, the temperature of step (a) and/or step (b) is from about 1° C. to about 100° C. As examples, the temperature is about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or about 100° C., and any range or value therebetween.

In several embodiments, the duration of step (b) is from about 1 minute to about 10 hours. As examples the duration is about 10 minutes, 20 minutes, 30 minutes, 40 minutes, 50 minutes, 60 minutes, 70 minutes, 80 minutes, 90 minutes, 2 hours, 3 hours, 4 hours, 5 hours, 6 hours, 7 hours, 8 hours, 9 hours, or about 10 hours, and any range or value therebetween.

In embodiments, the duration of step (b) is from about 1 minute to about 10 minutes. As examples the duration is about 1 minute, 2 minutes, 3 minutes, 4 minutes, 5 minutes, 6 minutes, 7 minutes, 8 minutes, 9 minutes, or about 10 minutes, and any range or value therebetween.

In numerous embodiments, the ratio (weight to weight or weight to volume or volume to volume) of the high salinity brine to pozzolan and/or a latently hydraulic material varies from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In various embodiments, the ratio (weight to weight or weight to volume or volume to volume) of the high salinity brine to CO₂ varies from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the pozzolan and/or a latently hydraulic material to CO₂ varies from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In some embodiments, a filler material or another additive is added to the combination of step (a). In some cases, the filler material or the other additive comprises sand, gravel, lightweight aggregate, or crushed stone, and a combination thereof.

In numerous embodiments, the ratio (weight to weight or weight to volume or volume to volume) of the aggregate to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the aggregate to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In many embodiments, the latently hydraulic material is a slag. In some cases, the slag is added in step (a) in addition to the added pozzolan. In alternate cases, the slag is added in step (a) instead of pozzolan. In some cases, the slag is added in step (a) instead of an activator.

In embodiments, the method further comprises isolating the cementitious aggregate composition from the reduced salinity water component or other liquid components. In some cases, the isolating comprises straining to capture the cementitious aggregate composition.

In various embodiments, the cementitious aggregate composition is molded into a shaped cementitious composition. Without wishing to be bound by theory, particles of the cementitious aggregate composition can stick to each other, thereby permitting the molding of the composition into shapes. In some case, molding may not require an additional binder to keep the molded shape and in other cases, molding may require an additional binder to keep the molded shape. In some cases, the shaped cementitious composition permits attachment by flora, e.g., algae, and/or fauna, e.g., coral, relative to a standard cement. In various cases, the shaped cementitious composition permits stronger attachment by flora and/or fauna relative to a standard cement and/or the shaped cementitious composition permits more plentiful attachment by flora and/or fauna relative to a standard cement. When molded, the shaped cementitious composition comprises gaps between particles of the aggregate such that the of the shaped cementitious composition is has a more porous surface, is more porous throughout its volume, and is less dense than a standard cement. The gaps of a shaped cementitious composition may be up to 500 nm in diameter. Additionally, a shaped cementitious composition may have up to 85% of the volume being void (e.g., the sum of all gaps) whereas a standard cement may have about 40% of the volume being void.

The shape of the shaped cementitious composition may be any shape capable of providing a substrate for aquatic flora and/or fauna attachment. The shape could be planar, circular, rounded, elongated, flat, rectangular, or any combination thereof. In some embodiments, the shape is pyramidal with closed surfaces. In other embodiments, the shape is pyramidal with open surfaces such that the shape comprises four bars or cylinders that form a square base and four bars or cylinders each originating at a corner of the square and converging to form the pyramid's apex.

The shaped cementitious aggregate composition be used in any way that any cement can be used.

In many embodiments, the cementitious composition further comprises at least one filler material or other additive, the at least one filler or other additive can be pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

In some embodiments, the ratio (weight to weight or weight to volume or volume to volume) of the filler material or other additive to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the filler material or other additive to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In another aspect, the present disclosure provides a cementitious aggregate composition comprising more CO₂ per gram and/or calcium, silicon, aluminum, magnesium and/or iron per gram than a standard cement. In some cases, the cementitious aggregate composition comprises up to 50% more CO₂ per gram than a standard cement. In various cases, the cementitious aggregate composition comprises up to 50%m ore calcium, silicon, aluminum, magnesium and/or iron per gram than a standard cement.

The cementitious aggregate composition is obtained by any herein-disclosed method, e.g., a method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a reduced salinity water component and a cementitious aggregate composition.

In some embodiments, the standard cement is a Portland Cement comprising cement clinker rather than a pozzolan.

In various embodiments, the standard cement comprises CaO, CaCO₃, SiO₂, Al₂O₃, Fe₂O₃, and CaSO₄·H₂O.

In many embodiments, the standard cement comprises a slaked or hydraulic dolomitic or calcareous lime blended with a natural or man-made pozzolanic or latently hydraulic material.

In numerous embodiments, the standard cement, e.g., a standard hydrated cement, is a Portland Cement comprising cement clinker rather than pozzolan.

In a further aspect, the present disclosure provides a cementitious aggregate composition comprising more numerous pores per unit volume and/or larger average pores than a standard cement. The cementitious aggregate composition is obtained by any herein-disclosed method, e.g., a method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a reduced salinity water component and a cementitious aggregate composition.

In some embodiments, the standard cement is a Portland Cement comprising cement clinker rather than a pozzolan.

In various embodiments, the standard cement comprises CaO, CaCO₃, SiO₂, Al₂O₃, Fe₂O₃, and CaSO₄·H₂O.

In many embodiments, the standard cement comprises a slaked or hydraulic dolomitic or calcareous lime blended with a natural or man-made pozzolanic or latently hydraulic material.

In numerous embodiments, the standard cement, e.g., a standard hydrated cement, is a Portland Cement comprising cement clinker rather than pozzolan.

In an additional aspect, the present disclosure provides a cementitious aggregate composition that is less dense per unit volume than a standard cement. The cementitious aggregate composition is obtained by any herein-disclosed method, e.g., a method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a reduced salinity water component and a cementitious aggregate composition.

In some embodiments, the standard cement is a Portland Cement comprising cement clinker rather than a pozzolan.

In various embodiments, the standard cement comprises CaO, CaCO₃, SiO₂, Al₂O₃, Fe₂O₃, and CaSO₄·H₂O.

In many embodiments, the standard cement comprises a slaked or hydraulic dolomitic or calcareous lime blended with a natural or man-made pozzolanic or latently hydraulic material.

In numerous embodiments, the standard cement, e.g., a standard hydrated cement, is a Portland Cement comprising cement clinker rather than pozzolan.

An additional aspect of the present disclosure is a shaped cementitious composition obtained by any herein-disclosed method. The cementitious aggregate composition is obtained by any herein-disclosed method, e.g., a method comprising steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a reduced salinity water component and a cementitious aggregate composition. The cementitious aggregate composition is molded into a shaped cementitious composition.

In various embodiments, the shaped cementitious composition permits attachment by flora, e.g., algae, and/or fauna, e.g., coral, relative to a standard cement. In various cases, the shaped cementitious composition permits stronger attachment by flora and/or fauna relative to a standard cement and/or the shaped cementitious composition permits more plentiful attachment by flora and/or fauna relative to a standard cement.

In some embodiments, a filler material or another additive is added to the combination of step (a). In some cases, the filler material or the other additive comprises sand, gravel, lightweight aggregate, or crushed stone, and a combination thereof.

In numerous embodiments, the ratio (weight to weight or weight to volume or volume to volume) of the aggregate to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the aggregate to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In many embodiments, the latently hydraulic material is a slag. In some cases, the slag is added in step (a) in addition to the added pozzolan. In alternate cases, the slag is added in step (a) instead of pozzolan. In some cases, the slag is added in step (a) instead of an activator.

In embodiments, the method further comprises isolating the cementitious aggregate composition from the reduced salinity water component or other liquid components. In some cases, the isolating comprises straining to capture the cementitious aggregate composition.

In various embodiments, the cementitious aggregate composition is molded into a shaped cementitious composition. Without wishing to be bound by theory, particles of the cementitious aggregate composition can stick to each other, thereby permitting the molding of the composition into shapes. In some case, molding may not require an additional binder to keep the molded shape and in other cases, molding may require an additional binder to keep the molded shape. In some cases, the shaped cementitious composition permits attachment by flora, e.g., algae, and/or fauna, e.g., coral, relative to a standard cement. In various cases, the shaped cementitious composition permits stronger attachment by flora and/or fauna relative to a standard cement and/or the shaped cementitious composition permits more plentiful attachment by flora and/or fauna relative to a standard cement. When molded, the shaped cementitious composition comprises gaps between particles of the aggregate such that the of the shaped cementitious composition is has a more porous surface, is more porous throughout its volume, and is less dense than a standard cement. The gaps of a shaped cementitious composition may be up to 500 nm in diameter. Additionally, a shaped cementitious composition may have up to 85% of the volume being void (e.g., the sum of all gaps) whereas a standard cement may have about 40% of the volume being void.

The shape of the shaped cementitious composition may be any shape capable of providing a substrate for aquatic flora and/or fauna attachment. The shape could be planar, circular, rounded, elongated, flat, rectangular, or any combination thereof. In some embodiments, the shape is pyramidal with closed surfaces. In other embodiments, the shape is pyramidal with open surfaces such that the shape comprises four bars or cylinders that form a square base and four bars or cylinders each originating at a corner of the square and converging to form the pyramid's apex.

In many embodiments, the cementitious composition further comprises at least one filler material or other additive, the at least one filler or other additive is selected from the following: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

In some embodiments, the ratio (weight to weight or weight to volume or volume to volume) of the filler material or other additive to each of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

In embodiments, the ratio (weight to weight or weight to volume or volume to volume) of the filler material or other additive to the combined weight or volume of the high salinity brine, CO₂, and the pozzolan and/or the latently hydraulic material from 1:100 to 100:1. As examples the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1 and any range or value therebetween.

The methods, steps, and/or features thereof disclosed in FIG. 1 or FIG. 3 to FIG. 5 may be used to produce a composition of any herein-disclosed aspect or embodiment.

In various embodiments, a mixture which is transformed into a cementitious aggregate composition and a reduced salinity water component, may comprise one of the following combinations:
(1) brine, (2) CO₂, and (3) a natural pozzolan;
(1) brine, (2) CO₂, (3) a natural pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a man-made pozzolan;
(1) brine, (2) CO₂, (3) a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a latently hydraulic material;
(1) brine, (2) CO₂, (3) a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan and a man-made pozzolan;
(1) brine, (2) CO₂, (3) a natural pozzolan and a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan and a latently hydraulic material;
(1) brine, (2) CO₂, (3) a natural pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a man-made pozzolan and a latently hydraulic material;
(1) brine, (2) CO₂, (3) a man-made pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO);
(1) brine, (2) CO₂, and (3) a natural pozzolan, a man-made pozzolan, and a latently hydraulic material; or
(1) brine, (2) CO₂, (3) a natural pozzolan, a man-made pozzolan, and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO).

In various cementitious aggregate compositions obtained by a tank precipitation method, an activator is preferably included in the combination.

Any composition or component thereof, or method for manufacturing a composition or component thereof, or reagent used in a method for manufacturing a composition or component thereof disclosed herein is applicable to any herein-disclosed composition, component, method or reagent. In other words, any aspect or embodiment described herein can be combined with any other aspect or embodiment as disclosed herein.

### Pozzolans

Without being bound by theory, particle size of the pozzolan(s) may affect reactivity of the material. For example, smaller particle size may provide increased reactivity. The pozzolan(s) may be provided at any particle size. In embodiments, at least about 90% of the particles of the pozzolan(s) fit through a mesh size of about #120 to about #1200. In embodiments, at least about 90% of the particles of the pozzolan(s) fit through a mesh size of about #120 to about #1000. In embodiments, at least about 90% of the particles of the pozzolan(s)fit through a mesh size of about #120 to about #800. In embodiments, at least about 90% of the particles of the pozzolan(s) fit through a mesh size of about #120 to about #600. In embodiments, at least about 90% of the particles of the pozzolan(s) fit through a mesh size of about #120 to about #400. In embodiments, at least about 90% of the particles of the pozzolan(s) fit through a mesh size of about #120 to about #325. In embodiments, at least about 90% of the particles of the pozzolan(s) fit through a mesh size of about #200 to about #1200. In embodiments, at least about 90% of the particles of the pozzolan(s) fit through a mesh size of about #200 to about #1000. In embodiments, at least about 90% of the particles of the pozzolan(s) fit through a mesh size of about #200 to about #800. In embodiments, at least about 90% of the particles of the pozzolan(s) fit through a mesh size of about #200 to about #700. In embodiments, at least about 90% of the particles of the pozzolan(s) fit through a mesh size of about #200 to about #600. In embodiments, at least about 90% of the particles of the pozzolan(s) fit through a mesh size of about #200 to about #500. In embodiments, at least about 90% of the particles of the pozzolan(s) fit through a mesh size of about #200 to about #400. In embodiments, at least about 90% of the particles of the pozzolan(s) fit through a mesh size of about #200 to about #325.

Mesh size can be any value or subrange within the recited ranges.

In embodiments, at least about 95% of the particles of the pozzolan(s) fit through a mesh size of about #120 to about #1200. In embodiments, at least about 96% of the particles of the pozzolan(s) fit through a mesh size of about #120 to about #1200. In embodiments, at least about 97% of the particles of the pozzolan(s) fit through a mesh size of about #120 to about #1200. In embodiments, at least about 98% of the particles of the pozzolan(s) fit through a mesh size of about #120 to about #1200. In embodiments, at least about 99% of the particles of the pozzolan(s) fit through a mesh size of about #120 to about #1200. In embodiments, at least about 98% of the particles of the pozzolan(s) fit through a mesh size of about #120 to about #1000. In embodiments, at least about 98% of the particles of the pozzolan(s) fit through a mesh size of about #120 to about #800. In embodiments, at least about 98% of the particles of the pozzolan(s) fit through a mesh size of about #120 to about #600. In embodiments, at least about 98% of the particles of the pozzolan(s) fit through a mesh size of about #120 to about #400. In embodiments, at least about 98% of the particles of the pozzolan(s) fit through a mesh size of about #120 to about #325. In embodiments, at least about 98% of the particles of the pozzolan(s) fit through a mesh size of about #200 to about #1200. In embodiments, at least about 98% of the particles of the pozzolan(s) fit through a mesh size of about #200 to about #1000. In embodiments, at least about 98% of the particles of the pozzolan(s) fit through a mesh size of about #200 to about #800. In embodiments, at least about 98% of the particles of the pozzolan(s) fit through a mesh size of about #200 to about #700. In embodiments, at least about 98% of the particles of the pozzolan(s) fit through a mesh size of about #200 to about #600. In embodiments, at least about 98% of the particles of the pozzolan(s) fit through a mesh size of about #200 to about #500. In embodiments, at least about 98% of the particles of the pozzolan(s) fit through a mesh size of about #200 to about #400. In embodiments, at least about 98% of the particles of the pozzolan(s) fit through a mesh size of about #200 to about #325. Mesh size can be any value or subrange within the recited ranges.

A pozzolan (either natural or man-made) may be or is a silicious or siliciious and aluminous material.

### Natural Pozzolans

A natural pozzolan is a raw pozzolan that is found in natural deposits. In embodiments, the natural pozzolan is not calcined. A material is referred to as “calcined” when it has been heated below the temperature of fusion to alter its composition or physical state.

Natural pozzolans have been used to replace cement clinker in the production of Portland Cement. Cement clinker is a solid material produced by sintering limestone and aluminosilicate material comprising four mineral phases: two calcium silicates, alite (Ca₃Si) and belite (Ca₂Si), tricalcium aluminate (Ca₃Al) and calcium aluminoferrite (Ca₄AlFe). The clinker is ground to a fine powder and used as the binder, where a small amount of gypsum must be added to avoid the flash setting of the tricalcium aluminate (Ca₃Al₂O₆), the most reactive mineral phase (exothermic hydration reaction) in Portland clinker.

In contrast, the reactive chemical composition of pozzolans and natural pozzolans may comprise, but not limited to, silica (SiO₂), alumina (Al₂O₃), and iron oxide (Fe₂O₃). Natural pozzolans encompass a broad range of materials that include, but are not limited to, volcanic rock (rhyolite, obsidian, pitchstone, pumice, basalt or trap, and andesite) volcanic ash, sedimentary clays and shales, calcined clays, rice husk ash, diatomaceous earth, metakaolin and olivine.

Despite the advantages and historical use of natural pozzolans in the production of Portland cement-based concrete as clinker alternatives, there are several obstacles or disadvantages: the use of pozzolans may reduce the early strength of concrete, making such cements unsuitable for precast applications and potentially increasing construction times. They also may increase water demand during concrete production and can lower resistance to carbonation, which raises the risk of corrosion to carbon (black) steel reinforcement. However, the materials and methods described herein provide improved alternatives.

In embodiments, the natural pozzolan used in a composition or method provided herein is selected from rhyolite, obsidian, pitchstone, pumice, basalt, andesite, volcanic ash, sedimentary clay, shale, wollastanite, opaline shale, diatomaceous earth, olivine, and combinations thereof. In embodiments, the natural pozzolan includes rhyolite. In embodiments, the natural pozzolan includes obsidian. In embodiments, the natural pozzolan includes pitchstone. In embodiments, the natural pozzolan includes pumice. In embodiments, the natural pozzolan includes basalt. In embodiments, the natural pozzolan includes andesite. In embodiments, the natural pozzolan includes volcanic ash. In embodiments, the natural pozzolan includes sedimentary clay. In embodiments, the natural pozzolan includes shale. In embodiments, the natural pozzolan includes wollastanite. In embodiments, the natural pozzolan includes diatomaceous earth. In embodiments, the natural pozzolan includes opaline shale. In embodiments, the natural pozzolan includes olivine.

In embodiments, the natural pozzolan used in a composition or method provided expressly excludes one or more of the natural pozzolans listed herein.

### Natural Pozzolans: Volcanic Rock

Rhyolite is a silica-rich volcanic rock, which has a fine-grain or glassy in texture. It is formed from magma rich in silica that is extruded from a volcanic vent to cool quickly on the surface rather than slowly in the subsurface. The mineral composition of rhyolite comprises quartz, sanidine, and plagioclase, with minor amounts of hornblende and biotite. Chemically, the composition of rhyolite generally comprises SiO₂ and an alkali metal oxide, such as K₂O and Na₂O.

Obsidian is also formed from extruded lava from a volcano that cools rapidly with minimal crystal growth (i.e., glassy or fine-grained.) Like rhyolite, obsidian is extremely rich in SiO₂ at about 70 wt. % or more and also includes MgO and Fe₂O₃. While obsidian is used for manufacturing, the uses are typically for cutting and piercing tools.

Pitchstone is a volcanic glass similar to obsidian, formed when extruded lava rapidly cools. Pitchstone has a similar chemical composition to both rhyolite and obsidian, with the amount of SiO2 ranging in the amount from about 70 wt. % to 75 wt. %. Pitchstone comprises minerals as quartz, alkali feldspar, and plagioclase, and in smaller amounts pyroxene and hornblende.

Pumice is a porous volcanic rock created when super-heated, highly pressurized rock is violently ejected from a volcano. Pumice typically has a porosity of approximately 64%-85% by volume. The mineral composition of pumice includes feldspar, augite, hornblend, and zircon. Pumice mainly comprises SiO₂, Al₂O₃, and minor amounts of other oxides such as FeO, Fe₂O₃, Na₂O, and K₂O.

Basalt is a fine-grained, extrusive igneous rock formed from the rapid cooling of low-viscosity lava rich in magnesium and iron. While basalt has a relatively lower amount of SiO₂ compared to other common igneous rocks, basalt generally has a composition of 45-52 wt. % SiO₂, 2-5 wt. % total alkalis, 0.5-2.0 wt. % TiO₂, 5-14% wt. %, and 14 wt. % Al₂O₃. Basalt may include additional components, including (without limitation) calcium oxide and/or magnesium oxide. Andesite is a fine-grained volcanic rock that forms as a result of the rapid cooling and solidification of lava due to the eruption of arc volcanoes or oozing fissures. Andesite contains sodium-rich plagioclase feldspar (Na,Ca)[(Si,Al)AlSi₂]O₈ and may contain (usually <20%) hornblende amphibole, biotite, pyroxene and quartz minerals. In addition, andesite compositions include 52-63% silicon dioxide as well as an alkali oxide content (e.g., Na₂O, K₂O) ranging from 0 to 7% w/w. Andesite compositions are referred to respectively as low-silica or high-silica andesites when they contain either 52-57% or 57-63% SiO₂.

Olivine is a magnesium iron silicate found abundantly in the earth's upper mantel as a dense aggregate. Olivine is chemically represented as (Mg,Fe)₂SiO₄. Generally, olivine is abundant in low-silica mafic and ultramafic igneous rocks.

### Man-Made Pozzolans

Other materials may have pozzolanic activity, including some man-made materials. In some embodiments, the compositions and methods described herein may utilize pozzolans, including non-natural (e.g., man-made) pozzolans. In embodiments, man-made pozzolans are calcined materials. A material is referred to as “calcined” when it has been heated below the temperature of fusion to alter its composition or physical state. In embodiments, man-made pozzolans are recycled materials from industry (e.g., GGBFS).

Non-limiting examples of man-made pozzolans include metakaolin, fly ash (e.g., Class C fly ash), silica fume, ground glass (e.g., ground waste glass), slag (e.g. ground-granulated blast-furnace slag, blast-furnace slag, steel-furnace slag, basic-oxygen-furnace slag, electric-arc-furnace slag, ladle slag, copper slag, steel slag, iron slag, lead slag, nickel slag, zinc slag, aluminum slag, slag from other metals), burned organic matter residues (e.g., rice husk ash or rice hull ash), expanded clay, expanded shale, and calcine clay, and combinations thereof.

In embodiments, the man-made pozzolan used in a composition or method provided includes metakaolin. In embodiments, the man-made pozzolan includes fly ash. In embodiments, the man-made pozzolan includes silica fume. In embodiments, the man-made pozzolan includes burned organic matter residue. In embodiments, the man-made pozzolan includes ground glass. In embodiments, the man-made pozzolan includes ground waste glass. In embodiments, the man-made pozzolan includes slag. In embodiments, the man-made pozzolan includes ground-granulated blast-furnace slag. In embodiments, the man-made pozzolan includes blast-furnace slag. In embodiments, the man-made pozzolan includes steel-furnace slag. In embodiments, the man-made pozzolan includes basic-oxygen-furnace slag. In embodiments, the man-made pozzolan includes electric-arc-furnace slag. In embodiments, the man-made pozzolan includes ladle slag. In embodiments, the man-made pozzolan includes copper slag. In embodiments, the man-made pozzolan includes steel slag. In embodiments, the man-made pozzolan includes iron slag. In embodiments, the man-made pozzolan includes lead slag. In embodiments, the man-made pozzolan includes nickel slag. In embodiments, the man-made pozzolan includes zinc slag. In embodiments, the man-made pozzolan includes aluminum slag. In embodiments, the man-made pozzolan includes slag from other metals. In embodiments, the man-made pozzolan includes burned organic matter residues. In embodiments, the man-made pozzolan includes rice husk ash (rice hull ash). In embodiments, the man-made pozzolan includes expanded clay. In embodiments, the man-made pozzolan includes expanded shale. In embodiments, the man-made pozzolan includes calcine clay.

In embodiments, the materials described herein may be chemically treated (e.g., with acid) prior to use. In embodiments, the materials described herein are not chemically treated prior to use.

In embodiments, the man-made pozzolan used in a composition or method provided expressly excludes one or more of the man-made pozzolans listed herein.

Where the term “pozzolan” is recited, it is to be understood that natural pozzolan, man-made pozzolan, or a mixture thereof is intended.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. In some cases, the term “about” refers to +10% of a stated number or value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The terms “increased”, “increasing”, “increase”, “improved”, “improvement”, “improving” and the like, are used herein to generally means an increase by a statically significant amount. In some aspects, the terms “increased” or “improved” means an increase or improvement of at least 10% as compared to a reference level, for example an increase or improvement of at least about 10%, at least about 20%, or at least about 30%, or at least about 40%, or at least about 50%, or at least about 60%, or at least about 70%, or at least about 80%, or at least about 90% or up to and including a 100%increase or any improvement between 10-100%as compared to a reference level, standard, or control. Other examples of “increase” or “improvement” includes an increase of at least 2-fold, at least 5-fold, at least 10-fold, at least 20-fold, at least 50-fold, at least 100-fold, at least 1000-fold or more as compared to a reference level.

The terms “decreased”, “decreasing”, “decrease”, “reduced”, “reducing”, “reduce” and the like, are used herein generally to mean a decrease or reduction by a statistically significant amount. In some aspects, “decreased” or “reduced” means a reduction by at least 10% as compared to a reference level, for example a decrease or reduction by at least about 20%, or at least about 30%, or at least about 40%, or at least about 50%, or at least about 60%, or at least about 70%, or at least about 80%, or at least about 90% or up to and including a 100% decrease or reduction (e.g., absent level or non-detectable level as compared to a reference level), or any decrease or reduction between 10-100% as compared to a reference level.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “accelerant” or “activator” is used in accordance with its plain ordinary meaning and refers a substance that improves the chemical reaction and affords a higher strength material. In embodiments, activators or accelerants contemplated in the present application include, but are not limited to, sulfates, nitrates, phosphates and chloride. In embodiments, the activator may be any sodium, lithium or potassium salt or hydroxide and any alkali or alkali earth metal ions and including Mg(OH)₂, MgO, Ca(OH)₂, CaCO₃, Al₂(SO₄)₃, and/or CaO. The amount of activator or accelerant added may vary depending on various factors. For example, more activator or accelerant may be added to large amounts of material (i.e., high mass). In addition, the lower the ambient temperature, the more activator or accelerant may be used. The amount of activator or accelerant used will vary depending on the amount of and/or type of pozzolan, MgO or Mg(OH)₂ used. The amount of activator or accelerant used will vary depending on the ratio of pozzolan, MgO or Mg(OH)2 used. The amount of activator or accelerant used will vary based on the aqueous solution (e.g., water, non-potable water, brackish water, brine, concentrated brine, seawater) used.

As used herein, the term “aggregates”, “aggregate”, “filler material” or “other additive” is used in accordance with its plain ordinary meaning and refers to inert granular materials such as sand, gravel, lightweight aggregate, or crushed stone whether normal weight and/or lightweight that, along with cementitious materials and other optional raw materials such as pigment and/or admixtures, are used in concrete. Further, the term “aggregates” as used herein can include ASTM International C 33 fine aggregates, ASTM International C 33 coarse aggregates, and other particulate materials mixed into a cementitious composition. The aggregate can be processed: crushed, screened, and washed to obtain proper cleanliness and gradation. In some cases, a beneficiation process such as jigging or heavy media separation can be used to upgrade the quality. Once processed, the aggregates can be handled and stored to minimize segregation and degradation and prevent contamination and to also protect from the weather as well as to allow to drain away and/or evaporate moisture. Aggregates, from different sources, or produced by different methods, may differ considerably in particle shape, size and texture. Shape of the aggregates of the present disclosure may be cubical and reasonably regular, essentially rounded, angular, or irregular. Surface texture may range from relatively smooth with small, exposed pores to irregular with small to large, exposed pores. Particle shape and surface texture of both fine and coarse aggregates may influence proportioning of mixtures in such factors as workability, pumpability, fine-to-coarse aggregate ratio, and water requirement.

As used herein, the term “aggregate-like, solid particle” refers to a solid particle that visually resembles sand. The particles contemplated herein range in size from about 0.0625 mm to about 40.0 mm. In embodiments, coarse aggregate sizes are larger than 4.75 mm, while fine aggregates are 4.75 mm or less. In embodiments, a maximum size up to 40 mm is used for coarse aggregate in most structural applications, while for mass concreting purposes such as dams, sizes up to 150 mm may be used. In embodiments, fine aggregates have particles up to a minimum size of 0.075 mm.

As used herein, the term “basalt” refers to a fine-grained, extrusive igneous rock formed from the rapid cooling of low-viscosity lava rich in magnesium and iron. While basalt has a relatively lower amount of SiO₂ compared to other common igneous rocks, basalt generally has a composition of 45-52 wt. % SiO₂, 2-5 wt. % total alkalis, 0.5-2.0 wt. % TiO2, 5-14% wt. %, and 14 wt. % Al₂O₃.

As used herein, the term “brine” is used in accordance with its plain ordinary meaning, and refers to a high concentration of salt in water. In embodiments, the concentration ranges from about 3 g of salt per liter of water to 26 g of salt per liter of water. In embodiments, the salt concentration of brine exceeds that of natural seawater. In embodiments, the salt concentration of brine is at least 101% greater than the salt concentration of natural seawater. In embodiments, the salt concentration of brine ranges from about 101% greater than the salt concentration of natural seawater to about 1000% greater than the salt concentration of natural seawater. In embodiments, brine is also referred to as desalination brine effluent. In embodiments, brine may include, but is not limited to, trace metals such as iron, nickel, chromium, and molybdenum. In embodiments, the brine is not processed. In embodiments, the brine is minimally processed. In embodiments, the brine is highly concentrated. In embodiments, the brine contains one or more reactants. In embodiments, the brine contains at least 75% (by weight) of one or more reactants. In embodiments, the brine is supplemented with one or more reactants.

As used herein, “bauxite” refers to a naturally occurring, heterogeneous material composed primarily of one or more aluminum hydroxide minerals plus various mixtures of silica, iron oxide, titania, aluminosilicate, and other impurities. In embodiments, bauxite residue which is an industrial wasted generated during the processing of bauxite into alumina using the Bayer process forms what is conventionally referred to as “red mud.”

As used herein, “calcined pozzolans” is used in accordance with its plain ordinary meaning and refers to products that are derived from shales and clays.

As used herein “calcium aluminates” refers to a range of materials that are obtained by combining calcium oxide and aluminum oxide in the presence of high temperatures. Calcium aluminates include tricalcium aluminate (3CaO·Al₂O₃), dodecacalcium hepta-aluminate (12CaO·7Al₂O₃) (C₁₂A₇) (mayenite), monocalcium aluminate (CaO·Al₂O₃) (CA) (occurring in nature as krotite and dmitryivanovite as two polymorphs), monocalcium 56ealuminate (CaO·2Al₂O₃) (CA₂) (occurring in nature as grossite), monocalcium hexa-aluminate (CaO·6Al₂O₃) (CA₆) (occurring in nature as hibonite).

Calcium aluminate cements refer to compositions that are sulfate free and afford hydrated calcium aluminates or carboaluminates. In embodiments, the major constituent and most reactive phase of calcium aluminate cements is monocalcium aluminate (CaO·Al₂O₃), which contain other calcium aluminates as well as a variety of less reactive phases. In embodiments, calcium aluminate cements are able to obtain more strength than ordinary Portland cement. In embodiments, a retarding admixture is used.

As used herein, the term “cement” is used in accordance with its plain ordinary meaning and refers to powdery substance made for use in making mortar or concrete. For example, cement can be a material that sets, hardens, and/or adheres to other materials to bind them to together, for example to make materials such as concrete. In embodiments, concrete is a mineral binder free of any organic compounds. In embodiments, the present application contemplates a Portland-cement free product. Some embodiments contemplate a reduced Portland Cement containing material with less than 90%, 80%, 70%, 60%, 50%, 40%, 30%, 25%, 20%, 15%, 10%, or 5% of Portland Cement; or any sub value or subrange between 0%and 90%. In embodiments, Portland Cement comprises calcium, silicon, aluminum, and iron. In embodiments, Portland Cement comprises CaO, CaCO₃, SiO₂, Al₂O₃, Fe₂O₃, and CaSO₄·H₂O. In embodiments, cement may be characterized as non-hydraulic or hydraulic cement. It should further be understood that “cementitious” can mean a material, including a material according to the embodiments described herein that has one or more of the characteristics or features of cement. In some embodiments, the cementitious composition does not comprise Portland cement.

As used herein, the term “concrete” is used in accordance with its plain ordinary meaning and refers to an artificial, stonelike material used for various structural purposes, made by mixing cement and various aggregates, as sand, pebbles, gravel, or shale, with water and allowing the cementitious composition to harden. In embodiments, the term “stonelike” refers to a material that visually, functionally and/or characteristically resembles stone, including when its hardened state. In embodiments, “concrete-replacement material” is interchangeably used with “artificial, stonelike material” throughout.

As used herein, the term “artificial, stonelike material” refers to a cementitious material used for various structural purposes or non-structural purposes (such as a slab, panel, paver or tile), made by mixing a cement alternative as contemplated herein and various aggregates, as sand, pebbles, gravel, lightweight aggregate, or shale, with water and allowing the cementitious composition to harden. In embodiments, the term “stonelike” refers to a material that visually resembles stone. In embodiments, “concrete-replacement material” is interchangeably used with “artificial, stonelike material” throughout.

As used herein, the term “desalination” is used in accordance with its plain ordinary meaning and refers to the process of removing salts or other minerals and contaminants from seawater, brackish water, well water, and wastewater effluent and it is an increasingly common solution to obtain fresh water for human consumption and for domestic/industrial utilization.

As used herein, the phrase “desalination wastewater” refers to reject brine from desalination. In embodiments, the process of removing salt from seawater to afford freshwater produces a highly concentrated brine as a by-product. The by-product is usually disposed of by discharging it back into the sea, a process that requires costly pumping systems and that must be managed carefully to prevent damage to marine ecosystems. This process, if not managed properly, disturbs the local water and sediment by introducing a multi-component waste and increasing temperature, which also endangers the marine organisms due to the residual chemicals mixed into the brine from the pre-treatment process.

As used herein, the term “freshwater” refers to water with a low dissolved salt concentration. In embodiments, freshwater does not include seawater and brackish water. In embodiments, freshwater may include, but is not limited to, frozen and meltwater in ice sheets, ice caps, glaciers, snowfields and icebergs, natural precipitation (e.g., rainfall, snowfall, hail, sleet). In embodiments, the salt concentration is less than 5%, less than 4%, less than 3%, less than 2%, and less than 1%, including sub-values in-between.

As used herein, the term “non-hydraulic cement” is used in accordance with its plain ordinary meaning and refers to cement that does not set in wet conditions or under water. In embodiments, non-hydraulic cement sets as it dries and reacts with CO₂ in the air. In embodiments, non-hydraulic cement is resistant to degradation by chemicals after setting.

As used herein, the term “hydraulic cement” is used in accordance with its plain ordinary meaning and refers to cement that sets in wet conditions due to a chemical reaction between the dry ingredients and water. In embodiments, the chemical reaction results in mineral hydrates that are either completely or nearly insoluble in water. In embodiments, hydraulic cement also refers to Portland Cement.

As used herein, the term “latently hydraulic material” is used in accordance with its plain ordinary meaning. In some cases, the latently hydraulic material is a slag, as used herein. In some cases, the latently hydraulic material is a natural pozzolan or a manmade pozzolan and which has latently hydraulic properties. A latently hydraulic material may be a material that will react with water to form a solid over an extended period of time.

As used herein, the term “mixing” is used in accordance with its plain ordinary meaning and refers to any form of mixing and may include milling or grinding of substances in solid form.

As used herein, the term “mortar” is used in accordance with its plain ordinary meaning and refers to a material composed of binder(s).

As used herein, the term “negative carbon dioxide-emitting concrete-replacement material” refers to a material that has a net positive CO₂ absorption as opposed to having a lower carbon footprint. In embodiments, the present application contemplates a material that produces carbon credits. In embodiments, the concrete-replacement material absorbs more carbon dioxide than is emitted.

As used herein, the term “seawater” is used in accordance with its plain ordinary meaning and refers to water from the sea or ocean. In embodiments, seawater includes various salts, dissolved inorganic (e.g., minerals) and organic compounds, and other particulates.

As used herein, the term “silane” is used in accordance with its plain ordinary meaning. In embodiments, silane is used as a coupling reagent between two dissimilar materials, which creates critical surface tension.

As used herein, the term “slag” is used in accordance with its plain ordinary meaning and means any type of slag and may be used interchangeably with “ground-granulated blast-furnace slag.” Ground-granulated blast-furnace slag (“GGBFS”) refers to a composition obtained by quenching molten iron slag (a by-product of iron and steel-making) from a blast furnace in water or steam, to produce a glassy, granular product that is then dried and ground into a fine powder. In embodiments, slag includes any by-product following the separation (e.g., via smelting) of a metal from its raw ore that has cementitious components and/or characteristics. In embodiments, slag includes, but is not limited to, arc furnace slag, foundry furnace slag, induction furnace slag, and the like. In general, furnace slag is a non-metallic by-product comprising silicates, calcium-alumina-silicates. Slag may include slag from any metal, for example and without limitation steel, iron, copper, nickel, lead, aluminum, and zinc. Without being bound to any one theory, the slags contemplated herein relate to the use as a binder, which provides hydraulicity. The hydraulicity in turn may modulate the compression strength of the material. As contemplated herein, the use of ground-granulated blast-furnace slag reduces iron waste disposal in landfills. In some embodiments, the slag satisfies the ASTM requirements.

As used herein, the term “slurry” is used in accordance with its plain ordinary meaning and refers to a mixture of denser solids suspended in liquid. In embodiments, “brine slurry” refers to desalinated water waste product.

As used herein, the term “structural component” refers to any vertical or horizontal load-bearing member of a structure which supports dead or live loads in addition to its own weight and includes, but is not limited to, a foundation, an exterior or interior load-bearing wall, a column, a column beam, a floor, and a roof structure.

As used herein, the term “mold” refers to any container or form used to give shape to the material. In embodiments, a mold includes a well.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

Any composition or method disclosed herein is applicable to any herein-disclosed composition or method. In other words, any aspect or embodiment described herein can be combined with any other aspect or embodiment as disclosed herein.

### Additional Embodiments

Embodiment 1: A process for producing aggregate-like solid particles comprising the steps: (a) flowing a brine slurry through a pipe; (b) adding a dry-mix composition into the pipe through an opening in the pipe; and, (c) mixing the brine slurry with the dry mix-composition via turbulent flow; to produce aggregate-like solid particles.

Embodiment 2: The process of Embodiment 1 further comprising adding a stream of CO₂.

Embodiment 3: The process of Embodiment 1 further comprising adding pressure.

Embodiment 4: The process of Embodiment 1, wherein the brine slurry comprises water, Mg(OH)₂, nitrate, sulfate, sodium, phosphates, and chloride.

Embodiment 5: The process of Embodiment 1, wherein the dry-mix composition comprises a natural pozzolan, a pozzolan, slag, ground-granulated blast-furnace slag, calcium aluminate, a calcined pozzolan, a calcined material, wastes from refining bauxite into alumina, and combinations thereof.

Embodiment 6: The process of Embodiment 1, wherein the dry-mix composition comprises ground-granulated blast-furnace slag.

Embodiment 7: The process of Embodiment 1, wherein the dry-mix composition comprises a natural pozzolan.

Embodiment 8: The process of Embodiment 1, wherein the dry-mix composition comprises ground-granulated blast-furnace slag and a natural pozzolan.

Embodiment 9: The process of Embodiments 7 or 8, wherein the natural pozzolan is selected from the group consisting of rhyolite, obsidian, pitchstone, pumice, basalt, andesite, volcanic ash, sedimentary clays and shales, calcined clays, rice husk ash, diatomaceous earth, metakaolin, olivine, and combinations thereof.

Embodiment 10: The process of Embodiment 1, wherein the dry-mix composition comprises ground-granulated blast-furnace slag and at least one aggregate.

Embodiment 11: The process of Embodiment 10, wherein the at least one aggregate is selected from sand, gravel, lightweight aggregate, crushed stone, and combinations thereof.

Embodiment 12: The process of Embodiment 1, wherein the aggregate-like solid particle has a diameter of about 0.0625 mm to about 40.0 mm.

Embodiment 13: The process of Embodiment 1, wherein the pipe is between 12 inches and 18 inches in diameter.

Embodiment 14: The process of Embodiment 1, wherein the brine slurry comprises Mg(OH)₂ and the dry-mix composition comprises ground-granulated blast-furnace slag.

Embodiment 15: The process of Embodiment 14, wherein the ratio of Mg(OH)₂ to ground-granulated blast-furnace slag is from 75:25 by wt. % to 25:75 by wt. %.

Embodiment 16: The process of Embodiment 14, wherein the ratio of Mg(OH)₂ to ground-granulated blast-furnace slag is from 70:30 by wt. % to 30:70 by wt. %.

Embodiment 17: The process of Embodiment 14, wherein the ratio of Mg(OH)₂ to ground-granulated blast-furnace slag is from 70:30 by wt. % to 30:70 by wt. %.

Embodiment 18: The process of Embodiment 14, wherein the ratio of Mg(OH)₂ to ground-granulated blast-furnace slag is from 70:30 by wt. % to 30:70 by wt. %.

Embodiment 19: The process of Embodiment 14, wherein the ratio of Mg(OH)₂ to ground-granulated blast-furnace slag is from 65:35 by wt. % to 35:65 by wt. %.

Embodiment 20: The process of Embodiment 14, wherein the ratio of Mg(OH)₂ to ground-granulated blast-furnace slag is from 60:40 by wt. % to 40:60 by wt. %.

Embodiment 21: The process of Embodiment 14, wherein the ratio of Mg(OH)₂ to ground-granulated blast-furnace slag is from 55:45 by wt. % to 45:55 by wt. %.

Embodiment 22: The process of Embodiment 14, wherein the ratio of Mg(OH)₂ to ground-granulated blast-furnace slag is about 50:50 by wt. %.

Embodiment 23: The process of Embodiment 1, wherein the brine slurry comprises MgO and the dry-mix composition comprises ground-granulated blast-furnace slag.

Embodiment 24: The process of Embodiment 23, wherein the ratio of MgO to ground-granulated blast-furnace slag is from 75:25 by wt. % to 25:75 by wt. %.

Embodiment 25: The process of Embodiment 23, wherein the ratio of MgO to ground-granulated blast-furnace slag is from 70:30 by wt. % to 30:70 by wt. %.

Embodiment 26: The process of Embodiment 23, wherein the ratio of MgO to ground-granulated blast-furnace slag is from 70:30 by wt. % to 30:70 by wt. %.

Embodiment 27: The process of Embodiment 23, wherein the ratio of MgO to ground-granulated blast-furnace slag is from 70:30 by wt. % to 30:70 by wt. %.

Embodiment 28: The process of Embodiment 23, wherein the ratio of MgO to ground-granulated blast-furnace slag is from 65:35 by wt. % to 35:65 by wt. %.

Embodiment 29: The process of Embodiment 23, wherein the ratio of MgO to ground-granulated blast-furnace slag is from 60:40 by wt. % to 40:60 by wt. %.

Embodiment 30: The process of Embodiment 23, wherein the ratio of MgO to ground-granulated blast-furnace slag is from 55:45 by wt. % to 45:55 by wt. %.

Embodiment 31: The process of Embodiment 23, wherein the ratio of MgO to ground-granulated blast-furnace slag is about 50:50 by wt. %.

Embodiment 32: The process of Embodiment 1 further comprising the step of allowing the aggregate-like solid particles to flow out of the pipe into a natural body of water.

Embodiment 33: The process of Embodiment 32, wherein the aggregate-like solid particles in the natural body of water promotes aquatic growth.

Embodiment 34: The process of Embodiment 32, wherein the aggregate-like solid particles in the natural body of water promotes coral growth.

Embodiment 35: The process of Embodiment 32, wherein the aggregate-like solid particles in the natural body of water forms an artificial reef structure.

Embodiment 36: The process of any one of Embodiments 1 to 35, wherein the aggregate-like solid particles are configured to absorb and retain carbon dioxide.

### EXAMPLES

### Example 1

In this example, a method for manufacturing a cementitious aggregate composition and a reduced salinity water component is described.

The method includes steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a cementitious aggregate composition and a reduced salinity water component.

The conditions sufficient to transform the combination into a cementitious aggregate composition and a reduced salinity water component in step (b) comprise time and applied pressure.

The high salinity brine, CO₂, and pozzolan and/or a latently hydraulic material are combined simultaneously or are combined sequentially and in any order.

The high salinity brine may be obtained from a desalination facility, is natural seawater, or an industrial brine.

A benefit of the present method is that the reduced salinity water component can be added to a natural or artificial body of water. The reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In some cases, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process can more efficiently be performed, since, at least in part, because the sulphates and chlorides have already been removed. The extracted sodium may be isolated into a pure sodium product.

The salt concentration of the high salinity brine is greater than or about equal to the salt concentration of seawater, whereas the salt concentration of the reduced salinity water component is less than or about equal to the salt concentration of seawater. Notably, the salt concentration of the reduced salinity water component is less than the high salinity brine. In various cases, the high salinity brine comprises water with a salt concentration (e.g., chloride, sulphate, and sodium) higher than 50 parts per thousand.

Steps (a) and/or (b) occurs in a location within the desalination facility, adjacent to the desalination facility, or downstream from the desalination facility. These steps can occur in a pipeline, a tank, or basin. When step (b) occurs in a pipeline, pressure applied to the pipeline utilizing a series of pipe reducers, which are mechanical devices used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

The pozzolan combined in step (a) comprises silica (SiO₂), alumina (Al₂O₃), and/or iron oxide (Fe₂O₃). The pozzolan may be a natural pozzolan or a man-made pozzolan. The pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

The CO₂ of step (a) is from industrial waste, from environmental sources, or from molecular capture. In some cases, the industrial waste relates to alcoholic fermentation or the burning of fossil fuels. The CO₂ of step (a) is provided in gaseous, solid, and/or as a supercritical fluid form or as a dissolved gas.

In some cases, step (a) further comprises an activator; the activator may be any sodium, lithium or potassium salt or hydroxide and any alkali or alkali earth metal ions and including one or more of Mg(OH)₂, MgO, Ca(OH)₂, CaCO₃, Al₂(SO₄)₃, and CaO.

Step (b) produces exothermic heat and this exothermic heat is transformable from thermal energy into electrical energy.

The pressure in step (b) is from about ambient (1 bar) to about 250 bar, e.g., from about 100 bar to about 250 bar or from about ambient (1 bar) to about 100 bar, and any range or value therebetween. The pressure in step (b) may be greater than about 250 bar. For example, the pressure in step (b) is greater than, 250 bar, 500 bar, 750 bar, 1000 bar, 1250 bar, 1500 bar, 1750 bar, 2000 bar, 2250 bar, 2500 bar, 2750 bar, 3000 bar, 3250 bar, 3500 bar, 3750 bar, 4000 bar, 4250 bar, 4500 bar, 4750 bar, 5000 bar and any range or value therebetween. The temperature of step (a) and/or step (b) is from about 1° C. to about 100° C., and any range or value therebetween. The duration of step (b) is from about 1 minute to about 10 hours, and any range or value therebetween.

The ratio (weight to weight or weight to volume or volume to volume) of the high salinity brine to pozzolan and/or a latently hydraulic material varies from 1:100 to 100:1, and any range or value therebetween. The ratio (weight to weight or weight to volume or volume to volume) of the high salinity brine to CO₂ varies from 1:100 to 100:1, and any range or value therebetween. The ratio (weight to weight or weight to volume or volume to volume) of the pozzolan and/or a latently hydraulic material to CO₂ varies from 1:100 to 100:1, and any range or value therebetween.

In some cases, an aggregate is added to the combination of step (a). The aggregate may comprise sand, gravel, lightweight aggregate, or crushed stone, and a combination thereof.

In various cases, the latently hydraulic material is a slag. The slag may be added in step (a) in addition to the added pozzolan or the slag is added in step (a) instead of pozzolan. The slag may be added in step (a) instead of an activator.

At least one filler material or other additive, may be added to the cementitious aggregate composition. Examples of a filler or another additive: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

The method may further comprise Isolating the cementitious aggregate composition from the reduced salinity water component or other liquid components. The isolating may comprise straining to capture the cementitious aggregate composition.

When forming cementitious aggregate composition, the CO₂ is chemically reacted to form a crystalline form of carbon and this crystalline form is absorbed into the cementitious aggregate composition.

The cementitious aggregate composition formed by the above-described method will have features as disclosed elsewhere herein, e.g., particle size, pore size, pore number, and so forth.

### Example 2

In this example, another method for manufacturing a cementitious aggregate composition and a reduced salinity water component is described, here, using an in-line conversion method.

The includes steps of: (a) combining (i) a high salinity brine, (ii) CO₂, and (iii) a natural pozzolan, a man-made pozzolan, and/or a latently hydraulic material; and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO) and (b) permitting the combination obtained in step (a) to persist under conditions sufficient to transform the combination into a cementitious aggregate composition and a reduced salinity water component, wherein the conditions in step (b) preferably comprises applied pressure and wherein steps (a) and/or (b) occur in a pipeline.

The high salinity brine, CO₂, and pozzolan and/or a latently hydraulic material are combined simultaneously or are combined sequentially and in any order.

The high salinity brine may be obtained from a desalination facility, is natural seawater, or an industrial brine.

A benefit of the present method is that the reduced salinity water component can be added to a natural or artificial body of water. The reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In some cases, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process can more efficiently be performed, since, at least in part, because the sulphates and chlorides have already been removed. The extracted sodium may be isolated into a pure sodium product.

The salt concentration of the high salinity brine is greater than or about equal to the salt concentration of seawater, whereas the salt concentration of the reduced salinity water component is less than or about equal to the salt concentration of seawater. Notably, the salt concentration of the reduced salinity water component is less than the high salinity brine. In various cases, the high salinity brine comprises water with a salt concentration (e.g., chloride, sulphate, and sodium) higher than 50 parts per thousand.

Steps (a) and/or (b) occurs in a location within the desalination facility, adjacent to the desalination facility, or downstream from the desalination facility. These steps can occur in a pipeline. When step (b) occurs in a pipeline, pressure applied to the pipeline utilizing a series of pipe reducers, which are mechanical devices used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

The pozzolan combined in step (a) comprises silica (SiO₂), alumina (Al₂O₃), and/or iron oxide (Fe₂O₃). The pozzolan may be a natural pozzolan or a man-made pozzolan. The pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

The CO₂ of step (a) is from industrial waste, from environmental sources, or from molecular capture. In some cases, the industrial waste relates to alcoholic fermentation or the burning of fossil fuels. The CO₂ of step (a) is provided in gaseous, solid, and/or as a supercritical fluid form or as a dissolved gas.

In some cases, step (a) further comprises an activator; the activator may be any sodium, lithium or potassium salt or hydroxide and any alkali or alkali earth metal ions and including one or more of Mg(OH)₂, MgO, Ca(OH)₂, CaCO₃, Al₂(SO₄)₃, and CaO.

Step (b) produces exothermic heat and this exothermic heat is transformable from thermal energy into electrical energy.

The pressure in step (b) is from about ambient (1 bar) to about 250 bar, e.g., from about 100 bar to about 250 bar or from about ambient (1 bar) to about 100 bar, and any range or value therebetween. The pressure in step (b) may be greater than about 250 bar. For example, the pressure in step (b) is greater than, 250 bar, 500 bar, 750 bar, 1000 bar, 1250 bar, 1500 bar, 1750 bar, 2000 bar, 2250 bar, 2500 bar, 2750 bar, 3000 bar, 3250 bar, 3500 bar, 3750 bar, 4000 bar, 4250 bar, 4500 bar, 4750 bar, 5000 bar and any range or value therebetween. The temperature of step (a) and/or step (b) is from about 1° C. to about 100° C., and any range or value therebetween. The duration of step (b) is from about 1 minute to about 10 hours, and any range or value therebetween.

The ratio (weight to weight or weight to volume or volume to volume) of the high salinity brine to pozzolan and/or a latently hydraulic material varies from 1:100 to 100:1, and any range or value therebetween. The ratio (weight to weight or weight to volume or volume to volume) of the high salinity brine to CO₂ varies from 1:100 to 100:1, and any range or value therebetween. The ratio (weight to weight or weight to volume or volume to volume) of the pozzolan and/or a latently hydraulic material to CO₂ varies from 1:100 to 100:1, and any range or value therebetween.

In some cases, an aggregate is added to the combination of step (a). The aggregate may comprise sand, gravel, lightweight aggregate, or crushed stone, and a combination thereof.

In various cases, the latently hydraulic material is a slag. The slag may be added in step (a) in addition to the added pozzolan or the slag is added in step (a) instead of pozzolan. The slag may be added in step (a) instead of an activator.

At least one filler material or other additive, may be added to the cementitious aggregate composition. Examples of a filler or another additive: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

The method may further comprise Isolating the cementitious aggregate composition from the reduced salinity water component or other liquid components. The isolating may comprise straining to capture the cementitious aggregate composition.

When forming cementitious aggregate composition, the CO₂ is chemically reacted to form a crystalline form of carbon and this crystalline form is absorbed into the cementitious aggregate composition.

The cementitious aggregate composition formed by the above-described method will have features as disclosed elsewhere herein, e.g., particle size, pore size, pore number, and so forth.

### Example 3

In this example, a further method for manufacturing a cementitious aggregate composition and a reduced salinity water component is described, here, using an in-line conversion method.

The method includes steps of: (a) combining (i) a high salinity brine, (ii) CO₂, and (iii) a pozzolan and/or a latently hydraulic material; and (b) applying pressure to the combination obtained in step (a) for an amount and duration sufficient to transform the combination into a cementitious aggregate composition and a reduced salinity water component, wherein the conditions in step (b) comprise applied pressure and wherein steps (a) and/or (b) occur in a pipeline.

The high salinity brine, CO₂, and pozzolan and/or a latently hydraulic material are combined simultaneously or are combined sequentially and in any order.

The high salinity brine may be obtained from a desalination facility, is natural seawater, or an industrial brine.

A benefit of the present method is that the reduced salinity water component can be added to a natural or artificial body of water. The reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In some cases, the reduced salinity water component is returned to the desalination plant for a further round of desalination into fresh water.

In some embodiments, the reduced salinity water component is further processed to extract sodium, sulphates, and/or chlorides and produces additional fresh water. The extracted sodium may be isolated into a pure sodium product. The extraction of sodium from the reduced salinity water component is more efficient since the sodium and chloride have substantially already been removed.

The salt concentration of the high salinity brine is greater than or about equal to the salt concentration of seawater, whereas the salt concentration of the reduced salinity water component is less than or about equal to the salt concentration of seawater. Notably, the salt concentration of the reduced salinity water component is less than the high salinity brine. In various cases, the high salinity brine comprises water with a salt concentration (e.g., chloride, sulphate, and sodium) higher than 50 parts per thousand.

Steps (a) and/or (b) occurs in a location within the desalination facility, adjacent to the desalination facility, or downstream from the desalination facility. These steps can occur in a pipeline. When step (b) occurs in a pipeline, pressure applied to the pipeline utilizing a series of pipe reducers, which are mechanical devices used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

The pozzolan combined in step (a) comprises silica (SiO₂), alumina (Al₂O₃), and/or iron oxide (Fe₂O₃). The pozzolan may be a natural pozzolan or a man-made pozzolan. The pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

The CO₂ of step (a) is from industrial waste, from environmental sources, or from molecular capture. In some cases, the industrial waste relates to alcoholic fermentation or the burning of fossil fuels. The CO₂ of step (a) is provided in gaseous, solid, and/or as a supercritical fluid form or as a dissolved gas.

Step (b) produces exothermic heat and this exothermic heat is transformable from thermal energy into electrical energy.

The pressure in step (b) is from about ambient (1 bar) to about 250 bar, e.g., from about 100 bar to about 250 bar or from about ambient (1 bar) to about 100 bar, and any range or value therebetween. The pressure in step (b) may be greater than about 250 bar. For example, the pressure in step (b) is greater than, 250 bar, 500 bar, 750 bar, 1000 bar, 1250 bar, 1500 bar, 1750 bar, 2000 bar, 2250 bar, 2500 bar, 2750 bar, 3000 bar, 3250 bar, 3500 bar, 3750 bar, 4000 bar, 4250 bar, 4500 bar, 4750 bar, 5000 bar and any range or value therebetween. The temperature of step (a) and/or step (b) is from about 1° C. to about 100° C., and any range or value therebetween. The duration of step (b) is from about 1 minute to about 10 hours, and any range or value therebetween.

The ratio (weight to weight or weight to volume or volume to volume) of the high salinity brine to pozzolan and/or a latently hydraulic material varies from 1:100 to 100:1, and any range or value therebetween. The ratio (weight to weight or weight to volume or volume to volume) of the high salinity brine to CO₂ varies from 1:100 to 100:1, and any range or value therebetween. The ratio (weight to weight or weight to volume or volume to volume) of the pozzolan and/or a latently hydraulic material to CO₂ varies from 1:100 to 100:1, and any range or value therebetween.

In some cases, an aggregate is added to the combination of step (a). The aggregate may comprise sand, gravel, lightweight aggregate, or crushed stone, and a combination thereof.

In various cases, the latently hydraulic material is a slag. The slag may be added in step (a) in addition to the added pozzolan or the slag is added in step (a) instead of pozzolan.

At least one filler material or other additive, may be added to the cementitious aggregate composition. Examples of a filler or another additive: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

The method may further comprise Isolating the cementitious aggregate composition from the reduced salinity water component or other liquid components. The isolating may comprise straining to capture the cementitious aggregate composition.

When forming cementitious aggregate composition, the CO₂ is chemically reacted to form a crystalline form of carbon and this crystalline form is absorbed into the cementitious aggregate composition.

The cementitious aggregate composition formed by the above-described method will have features as disclosed elsewhere herein, e.g., particle size, pore size, pore number, and so forth.

### Example 4

In this example, a further method for manufacturing a cementitious aggregate composition and a reduced salinity water component is described, here, using an in-line conversion method.

An additional aspect of the present disclosure is an in-line conversion method comprising steps of: (a) combining (i) a high salinity brine, (ii) CO₂, and (iii) a pozzolan and/or a latently hydraulic material; (b) allowing the combination obtained in step (a) to persist for duration sufficient to transform the combination into a cementitious aggregate composition and a reduced salinity water component, wherein steps (a) and/or (b) occur in a pipeline.

The conditions sufficient to transform the combination into a cementitious aggregate composition and a reduced salinity water component in step (b) comprise time and applied pressure.

The high salinity brine, CO₂, and pozzolan and/or a latently hydraulic material are combined simultaneously or are combined sequentially and in any order.

The high salinity brine may be obtained from a desalination facility, is natural seawater, or an industrial brine.

A benefit of the present method is that the reduced salinity water component can be added to a natural or artificial body of water. The reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In some cases, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process can more efficiently be performed, since, at least in part, because the sulphates and chlorides have already been removed. The extracted sodium may be isolated into a pure sodium product.

The salt concentration of the high salinity brine is greater than or about equal to the salt concentration of seawater, whereas the salt concentration of the reduced salinity water component is less than or about equal to the salt concentration of seawater. Notably, the salt concentration of the reduced salinity water component is less than the high salinity brine. In various cases, the high salinity brine comprises water with a salt concentration (e.g., chloride, sulphate, and sodium) higher than 50 parts per thousand.

Steps (a) and/or (b) occurs in a location within the desalination facility, adjacent to the desalination facility, or downstream from the desalination facility. These steps can occur in a pipeline. When step (b) occurs in a pipeline, pressure applied to the pipeline utilizing a series of pipe reducers, which are mechanical devices used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

The pozzolan combined in step (a) comprises silica (SiO₂), alumina (Al₂O₃), and/or iron oxide (Fe₂O₃). The pozzolan may be a natural pozzolan or a man-made pozzolan. The pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

The CO₂ of step (a) is from industrial waste, from environmental sources, or from molecular capture. In some cases, the industrial waste relates to alcoholic fermentation or the burning of fossil fuels. The CO₂ of step (a) is provided in gaseous, solid, and/or as a supercritical fluid form or as a dissolved gas.

Step (b) produces exothermic heat and this exothermic heat is transformable from thermal energy into electrical energy.

The pressure in step (b) is from about ambient (1 bar) to about 250 bar, e.g., from about 100 bar to about 250 bar or from about ambient (1 bar) to about 100 bar, and any range or value therebetween. The pressure in step (b) may be greater than about 250 bar. For example, the pressure in step (b) is greater than, 250 bar, 500 bar, 750 bar, 1000 bar, 1250 bar, 1500 bar, 1750 bar, 2000 bar, 2250 bar, 2500 bar, 2750 bar, 3000 bar, 3250 bar, 3500 bar, 3750 bar, 4000 bar, 4250 bar, 4500 bar, 4750 bar, 5000 bar and any range or value therebetween. The temperature of step (a) and/or step (b) is from about 1° C. to about 100° C., and any range or value therebetween. The duration of step (b) is from about 1 minute to about 10 hours, and any range or value therebetween.

The ratio (weight to weight or weight to volume or volume to volume) of the high salinity brine to pozzolan and/or a latently hydraulic material varies from 1:100 to 100:1, and any range or value therebetween. The ratio (weight to weight or weight to volume or volume to volume) of the high salinity brine to CO₂ varies from 1:100 to 100:1, and any range or value therebetween. The ratio (weight to weight or weight to volume or volume to volume) of the pozzolan and/or a latently hydraulic material to CO₂ varies from 1:100 to 100:1, and any range or value therebetween.

In some cases, an aggregate is added to the combination of step (a). The aggregate may comprise sand, gravel, lightweight aggregate, or crushed stone, and a combination thereof.

In various cases, the latently hydraulic material is a slag. The slag may be added in step (a) in addition to the added pozzolan or the slag is added in step (a) instead of pozzolan. The slag may be added in step (a) instead of an activator.

At least one filler material or other additive, may be added to the cementitious aggregate composition. Examples of a filler or another additive: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

The method may further comprise Isolating the cementitious aggregate composition from the reduced salinity water component or other liquid components. The isolating may comprise straining to capture the cementitious aggregate composition.

When forming cementitious aggregate composition, the CO₂ is chemically reacted to form a crystalline form of carbon and this crystalline form is absorbed into the cementitious aggregate composition.

The cementitious aggregate composition formed by the above-described method will have features as disclosed elsewhere herein, e.g., particle size, pore size, pore number, and so forth.

### Example 5

In this example, yet another method for manufacturing a cementitious aggregate composition and a reduced salinity water component is described, here, using an in-line conversion method. The method includes steps of: (a) combining (i) a high salinity brine, (ii) CO₂, (iii) a pozzolan and/or a latently hydraulic material, and (iv) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); and (b) applying pressure to the combination obtained in step (a) for an amount and duration sufficient to transform the combination into a cementitious aggregate composition and a reduced salinity water component, wherein the conditions in step (b) comprise applied pressure and wherein steps (a) and/or (b) occur in a pipeline.

The high salinity brine, CO₂, and pozzolan and/or a latently hydraulic material are combined simultaneously or are combined sequentially and in any order.

The high salinity brine may be obtained from a desalination facility, is natural seawater, or an industrial brine.

A benefit of the present method is that the reduced salinity water component can be added to a natural or artificial body of water. The reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In some cases, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process can more efficiently be performed, since, at least in part, because the sulphates and chlorides have already been removed. The extracted sodium may be isolated into a pure sodium product.

The salt concentration of the high salinity brine is greater than or about equal to the salt concentration of seawater, whereas the salt concentration of the reduced salinity water component is less than or about equal to the salt concentration of seawater. Notably, the salt concentration of the reduced salinity water component is less than the high salinity brine. In various cases, the high salinity brine comprises water with a salt concentration (e.g., chloride, sulphate, and sodium) higher than 50 parts per thousand.

Steps (a) and/or (b) occurs in a location within the desalination facility, adjacent to the desalination facility, or downstream from the desalination facility. These steps can occur in a pipeline. When step (b) occurs in a pipeline, pressure applied to the pipeline utilizing a series of pipe reducers, which are mechanical devices used to reduce the diameter of a pipe, which creates pressure by restricting the flow of a fluid. In other cases, the pressure in step (b) is applied using another method which creates pressure sufficient to make the conversion process (e.g., chemical reactions) more efficient. In numerous cases, the applied pressure further reduces the amount of energy required and/or waste CO₂ produced.

The pozzolan combined in step (a) comprises silica (SiO₂), alumina (Al₂O₃), and/or iron oxide (Fe₂O₃). The pozzolan may be a natural pozzolan or a man-made pozzolan. The pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

The CO₂ of step (a) is from industrial waste, from environmental sources, or from molecular capture. In some cases, the industrial waste relates to alcoholic fermentation or the burning of fossil fuels. The CO₂ of step (a) is provided in gaseous, solid, and/or as a supercritical fluid form or as a dissolved gas.

In some cases, step (a) further comprises an activator; the activator may be any sodium, lithium or potassium salt or hydroxide and any alkali or alkali earth metal ions and including one or more of Mg(OH)₂, MgO, Ca(OH)₂, CaCO₃, Al₂(SO₄)₃, and CaO.

Step (b) produces exothermic heat and this exothermic heat is transformable from thermal energy into electrical energy.

The pressure in step (b) is from about ambient (1 bar) to about 250 bar, e.g., from about 100 bar to about 250 bar or from about ambient (1 bar) to about 100 bar, and any range or value therebetween. The pressure in step (b) may be greater than about 250 bar. For example, the pressure in step (b) is greater than, 250 bar, 500 bar, 750 bar, 1000 bar, 1250 bar, 1500 bar, 1750 bar, 2000 bar, 2250 bar, 2500 bar, 2750 bar, 3000 bar, 3250 bar, 3500 bar, 3750 bar, 4000 bar, 4250 bar, 4500 bar, 4750 bar, 5000 bar and any range or value therebetween. The temperature of step (a) and/or step (b) is from about 1° C. to about 100° C., and any range or value therebetween. The duration of step (b) is from about 1 minute to about 10 hours, and any range or value therebetween.

The ratio (weight to weight or weight to volume or volume to volume) of the high salinity brine to pozzolan and/or a latently hydraulic material varies from 1:100 to 100:1, and any range or value therebetween. The ratio (weight to weight or weight to volume or volume to volume) of the high salinity brine to CO₂ varies from 1:100 to 100:1, and any range or value therebetween. The ratio (weight to weight or weight to volume or volume to volume) of the pozzolan and/or a latently hydraulic material to CO₂ varies from 1:100 to 100:1, and any range or value therebetween.

In some cases, an aggregate is added to the combination of step (a). The aggregate may comprise sand, gravel, lightweight aggregate, or crushed stone, and a combination thereof.

In various cases, the latently hydraulic material is a slag. The slag may be added in step (a) in addition to the added pozzolan or the slag is added in step (a) instead of pozzolan. The slag may be added in step (a) instead of an activator.

At least one filler material or other additive, may be added to the cementitious aggregate composition. Examples of a filler or another additive: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

The method may further comprise Isolating the cementitious aggregate composition from the reduced salinity water component or other liquid components. The isolating may comprise straining to capture the cementitious aggregate composition.

When forming cementitious aggregate composition, the CO₂ is chemically reacted to form a crystalline form of carbon and this crystalline form is absorbed into the cementitious aggregate composition.

The cementitious aggregate composition formed by the above-described method will have features as disclosed elsewhere herein, e.g., particle size, pore size, pore number, and so forth.

### Example 6

In this example, yet another method for manufacturing a cementitious aggregate composition and a reduced salinity water component is described, here, using a tank precipitation method. The method including steps of: (a) combining (i) a high salinity brine, (ii) CO₂, and (iii) a pozzolan and/or a latently hydraulic material; and (b) permitting the combination obtained in step (a) to persist under conditions sufficient to transform the combination into a cementitious aggregate composition and a reduced salinity water component, wherein the conditions in step (b) does not comprise applied pressure and wherein steps (a) and/or (b) occur in a tank or basin. The conditions sufficient to transform the combination into a cementitious aggregate composition and a reduced salinity water component in step (b) comprise time and heat.

The high salinity brine, CO₂, and pozzolan and/or a latently hydraulic material are combined simultaneously or are combined sequentially and in any order.

The high salinity brine may be obtained from a desalination facility, is natural seawater, or an industrial brine.

A benefit of the present method is that the reduced salinity water component can be added to a natural or artificial body of water. The reduced salinity water component does less harm to flora and/or fauna present in the natural or artificial body of water relative to the harm that would be caused when the high salinity brine is added to a natural or artificial body of water. Without wising to be bound by theory, the reduced salinity water comprises less sulphates and chlorides than high salinity brine, which also are harmful to flora and/or fauna present in the natural or artificial body of water.

In some cases, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process can more efficiently be performed, since, at least in part, because the sulphates and chlorides have already been removed. The extracted sodium may be isolated into a pure sodium product.

The salt concentration of the high salinity brine is greater than or about equal to the salt concentration of seawater, whereas the salt concentration of the reduced salinity water component is less than or about equal to the salt concentration of seawater. Notably, the salt concentration of the reduced salinity water component is less than the high salinity brine. In various cases, the high salinity brine comprises water with a salt concentration (e.g., chloride, sulphate, and sodium) higher than 50 parts per thousand.

Steps (a) and/or (b) occurs in a location within the desalination facility, adjacent to the desalination facility, or downstream from the desalination facility.

The pozzolan combined in step (a) comprises silica (SiO₂), alumina (Al₂O₃), and/or iron oxide (Fe₂O₃). The pozzolan may be a natural pozzolan or a man-made pozzolan. The pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

The CO₂ of step (a) is from industrial waste, from environmental sources, or from molecular capture. In some cases, the industrial waste relates to alcoholic fermentation or the burning of fossil fuels. The CO₂ of step (a) is provided in gaseous, solid, and/or as a supercritical fluid form or as a dissolved gas.

Step (b) produces exothermic heat and this exothermic heat is transformable from thermal energy into electrical energy.

The temperature of step (a) and/or step (b) is from about 1° C. to about 100° C., and any range or value therebetween. The duration of step (b) is from about 1 minute to about 10 hours, and any range or value therebetween.

The ratio (weight to weight or weight to volume or volume to volume) of the high salinity brine to pozzolan and/or a latently hydraulic material varies from 1:100 to 100:1, and any range or value therebetween. The ratio (weight to weight or weight to volume or volume to volume) of the high salinity brine to CO₂ varies from 1:100 to 100:1, and any range or value therebetween. The ratio (weight to weight or weight to volume or volume to volume) of the pozzolan and/or a latently hydraulic material to CO₂ varies from 1:100 to 100:1, and any range or value therebetween.

In some cases, an aggregate is added to the combination of step (a). The aggregate may comprise sand, gravel, lightweight aggregate, or crushed stone, and a combination thereof.

In various cases, the latently hydraulic material is a slag. The slag may be added in step (a) in addition to the added pozzolan or the slag is added in step (a) instead of pozzolan.

At least one filler material or other additive, may be added to the cementitious aggregate composition. Examples of a filler or another additive: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

The method may further comprise Isolating the cementitious aggregate composition from the reduced salinity water component or other liquid components. The isolating may comprise straining to capture the cementitious aggregate composition.

When forming cementitious aggregate composition, the CO₂ is chemically reacted to form a crystalline form of carbon and this crystalline form is absorbed into the cementitious aggregate composition.

The cementitious aggregate composition formed by the above-described method will have features as disclosed elsewhere herein, e.g., particle size, pore size, pore number, and so forth.

### Example 7

In this example, yet another method for manufacturing a cementitious aggregate composition and a reduced salinity water component is described, here, using a tank precipitation method. The method includes steps of: (a) combining (i) a high salinity brine, (ii) CO₂, and (iii) a natural pozzolan, a man-made pozzolan, and/or a latently hydraulic material; and, optionally, an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO) and (b) permitting the combination obtained in step (a) to persist under conditions sufficient to transform the combination into a cementitious aggregate composition and a reduced salinity water component, wherein the conditions in step (b) does not comprise applied pressure and wherein steps (a) and/or (b) occur in a tank or basin.

The conditions sufficient to transform the combination into a cementitious aggregate composition and a reduced salinity water component in step (b) comprise time and heat.

The high salinity brine, CO₂, and pozzolan and/or a latently hydraulic material are combined simultaneously or are combined sequentially and in any order.

The high salinity brine may be obtained from a desalination facility, is natural seawater, or an industrial brine.

A benefit of the present method is that the reduced salinity water component can be added to a natural or artificial body of water. The reduced salinity water component does less harm to flora and/or fauna present in the natural or artificial body of water relative to the harm that would be caused when the high salinity brine is added to a natural or artificial body of water.

Without wising to be bound by theory, the reduced salinity water comprises less sulphates and chlorides than high salinity brine, which also are harmful to flora and/or fauna present in the natural or artificial body of water.

In some cases, the reduced salinity water component is returned to the desalination plant for a further round of desalination into fresh water.

In some embodiments, the reduced salinity water component is further processed to extract sodium to produce additional fresh water. In addition to or in combination with or after the above-described processes, a chloralkali process can more efficiently be performed, since, at least in part, because the sulphates and chlorides have already been removed. The extracted sodium may be isolated into a pure sodium product.

The salt concentration of the high salinity brine is greater than or about equal to the salt concentration of seawater, whereas the salt concentration of the reduced salinity water component is less than or about equal to the salt concentration of seawater. Notably, the salt concentration of the reduced salinity water component is less than the high salinity brine. In various cases, the high salinity brine comprises water with a salt concentration (e.g., chloride, sulphate, and sodium) higher than 50 parts per thousand.

Steps (a) and/or (b) occurs in a location within the desalination facility, adjacent to the desalination facility, or downstream from the desalination facility.

The pozzolan combined in step (a) comprises silica (SiO₂), alumina (Al₂O₃), and/or iron oxide (Fe₂O₃). The pozzolan may be a natural pozzolan or a man-made pozzolan. The pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

The CO₂ of step (a) is from industrial waste, from environmental sources, or from molecular capture. In some cases, the industrial waste relates to alcoholic fermentation or the burning of fossil fuels. The CO₂ of step (a) is provided in gaseous, solid, and/or as a supercritical fluid form or as a dissolved gas.

In some cases, step (a) further comprises an activator; the activator may be any sodium, lithium or potassium salt or hydroxide and any alkali or alkali earth metal ions and including one or more of Mg(OH)₂, MgO, Ca(OH)₂, CaCO₃, Al₂(SO₄)₃, and CaO.

Step (b) produces exothermic heat and this exothermic heat is transformable from thermal energy into electrical energy.

The temperature of step (a) and/or step (b) is from about 1° C. to about 100° C., and any range or value therebetween. The duration of step (b) is from about 1 minute to about 10 hours, and any range or value therebetween.

The ratio (weight to weight or weight to volume or volume to volume) of the high salinity brine to pozzolan and/or a latently hydraulic material varies from 1:100 to 100:1, and any range or value therebetween. The ratio (weight to weight or weight to volume or volume to volume) of the high salinity brine to CO₂ varies from 1:100 to 100:1, and any range or value therebetween. The ratio (weight to weight or weight to volume or volume to volume) of the pozzolan and/or a latently hydraulic material to CO₂ varies from 1:100 to 100:1, and any range or value therebetween.

In some cases, an aggregate is added to the combination of step (a). The aggregate may comprise sand, gravel, lightweight aggregate, or crushed stone, and a combination thereof.

In various cases, the latently hydraulic material is a slag. The slag may be added in step (a) in addition to the added pozzolan or the slag is added in step (a) instead of pozzolan. The slag may be added in step (a) instead of an activator.

At least one filler material or other additive, may be added to the cementitious aggregate composition. Examples of a filler or another additive: pumice or other volcanic rock or material, sand, gravel, crushed stone, aggregate (e.g., fine aggregate, coarse aggregate, intermediate aggregate, other types of aggregate, etc.), lightweight aggregate, talc, other clay material, fibers (e.g., steel and/or other metallic fibers, polypropylene and/or other polymeric fibers, glass fibers, asbestos fibers, carbon fibers, organic fibers, etc.), glass fiber reinforced plastic (GFRP), other reinforced polymers, admixtures or other additives that facilitate with fire protection, water protection, corrosion resistance/inhibition, workability, and/or one more other properties of the final cured product (e.g., MasterPel, RheoCell, MasterCell, etc.), sodium naphthalene sulfonate formaldehyde (SNF) and/or other surfactants, plasticizers, pigments, dyes and other color additives, titanium dioxide, other minerals, other natural or synthetic materials, other filler materials and/or the like.

The method may further comprise Isolating the cementitious aggregate composition from the reduced salinity water component or other liquid components. The isolating may comprise straining to capture the cementitious aggregate composition.

When forming cementitious aggregate composition, the CO₂ is chemically reacted to form a crystalline form of carbon and this crystalline form is absorbed into the cementitious aggregate composition.

The cementitious aggregate composition formed by the above-described method will have features as disclosed elsewhere herein, e.g., particle size, pore size, pore number, and so forth.

### Example 8

In this example, cementitious aggregate composition deposited into a natural or artificial body of water.

The method comprises obtaining a cementitious aggregate composition formed by a herein-disclosed method, e.g., from Example 1 to Example 7.

The cementitious aggregate composition may be filtered from the reduced salinity water component or allowed to sediment. The cementitious aggregate composition may be isolated on land and transported to a natural or artificial body of water.

The cementitious aggregate composition is deposited into natural or artificial body of water. There, the cementitious aggregate composition remains where deposited or a portion of the cementitious aggregate composition is distributed away from the deposit site, e.g., by water movement or currents, erosion, or animal activity.

Once deposited, the cementitious aggregate composition permits attachment by flora, e.g., algae, and/or fauna, e.g., coral. In many cases, the cementitious aggregate composition permits a preferred level of colonization by flora and/or fauna relative to a standard cement and/or the cementitious aggregate composition permits more plentiful attachment by flora and/or fauna relative to a standard cement.

### Example 9

In this example, a cementitious aggregate composition is molded into a shaped cementitious composition.

The method comprises obtaining a cementitious aggregate composition formed by a herein-disclosed method, e.g., from Example 1 to Example 7.

The cementitious aggregate composition is molded into a shaped cementitious composition. Particles of the cementitious aggregate composition can stick to each other, thereby permitting the molding of the composition into shapes. In some case, molding may not require an additional binder to keep the molded shape and in other cases, molding may require an additional binder to keep the molded shape. In some cases, the shaped cementitious composition, when deposited into a natural or artificial body of water, permits attachment by flora, e.g., algae, and/or fauna, e.g., coral, relative to a standard cement. In various cases, the shaped cementitious composition permits stronger attachment by flora and/or fauna relative to a standard cement and/or the shaped cementitious composition permits more plentiful attachment by flora and/or fauna relative to a standard cement. When molded, the shaped cementitious composition comprises gaps between particles of the aggregate such that the of the shaped cementitious composition is has a more porous surface, is more porous throughout its volume, and is less dense than a standard cement. The gaps of a shaped cementitious composition may be up to 500 nm in diameter. Additionally, a shaped cementitious composition may have up to 85% of the volume being void (e.g., the sum of all gaps) whereas a standard cement may have about 40% of the volume being void.

The shape of the shaped cementitious composition may be any shape capable of providing a substrate for aquatic flora and/or fauna attachment. The shape could be planar, circular, rounded, elongated, flat, rectangular, or any combination thereof. In some embodiments, the shape is pyramidal with closed surfaces. In other embodiments, the shape is pyramidal with open surfaces such that the shape comprises four bars or cylinders that form a square base and four bars or cylinders each originating at a corner of the square and converging to form the pyramid's apex.

Although the foregoing embodiments have been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method comprising steps of:
combining (i) a high salinity brine, (ii) CO₂, and (iii) a pozzolan and/or a latently hydraulic material; and
permitting the combination obtained in step (a) to persist under conditions sufficient to transform the combination into a cementitious aggregate composition and a reduced salinity water component.

2. The method of claim 1, wherein the conditions sufficient to transform the combination into a cementitious aggregate composition and a reduced salinity water component in step (b) comprise time and applied pressure or heat.

3. The method of claim 1, wherein the high salinity brine, CO₂, and pozzolan and/or a latently hydraulic material are combined simultaneously or sequentially.

4.-6. (canceled)

7. The method of claim 1, wherein the high salinity brine is obtained from a desalination facility, is natural seawater, or is an industrial brine.

8. (canceled)

9. The method of claim 1, wherein the salt concentration of the reduced salinity water component is less than or about equal to the salt concentration of seawater and/or is less than or about equal to the salt concentration of the high salinity brine.

10.-11. (canceled)

12. The method of claim 1, wherein the method further comprises further-adding an activator in step (a), the activator selected from sodium, lithium or potassium salt or hydroxide and any alkali or alkali earth metal ions.

13. (canceled)

14. The method of claim 12, wherein the activator is one or more of Mg(OH)₂, MgO, Ca(OH)₂, CaCO₃, Al₂(SO₄)₃, and CaO.

15. The method of claim 1, wherein the high salinity brine of step (a) has reduced amounts of sodium, potassium, chloride, sulphate, magnesium, and/or calcium ions via chemical precipitation, electrochemical methods, ion selective membranes, reverse osmosis, and/or selective precipitation by pH.

16. (canceled)

17. The method of claim 15, wherein the chemical precipitation occurs before step (a) and/or during step (a).

18. The method of claim 1, wherein the reduced salinity water component is added to a natural or artificial body of water or wherein the reduced salinity water component is further processed by a desalination facility to extract sodium and produce fresh water.

19. The method of claim 18, wherein the reduced salinity water component does less harm to flora and/or fauna present in the natural or artificial body of water relative to the harm that would be caused when the high salinity brine is added to a natural or artificial body of water.

20. The method of claim 19, wherein the reduced salinity water comprises less sulphates and chlorides than the high salinity brine, which are harmful to flora and/or fauna present in the natural or artificial body of water.

21.-23. (canceled)

24. The method of claim 7, wherein the method permits production by a desalination facility of a brine having a higher salt concentration relative to a method where the desalination facility produces brine as wastewater to be added to a natural or artificial body of water and/or permits production of more fresh water by a desalination facility relative to a method where the desalination facility produces brine as wastewater to be added to a natural or artificial body of water.

25.-29. (canceled)

30. The method of claim 1, wherein step (a) and/or step (b) occurs in a pipeline within a desalination facility, adjacent to a desalination facility, or downstream from the a desalination facility.

31.-33. (canceled)

34. The method of claim 1, wherein steps (a) and/or step (b) occurs in a tank or a basin.

35.-38. (canceled)

39. The method of claim 1, wherein the pozzolan is a natural pozzolan or a man-made pozzolan.

40. The method of claim 39, wherein the pozzolan comprises or is obtained from one or more of volcanic rock (e.g., rhyolite, obsidian, pitchstone, pumice, basalt or trap, or andesite); volcanic ash; sedimentary clays or shales' calcined clays; rice husk ash; diatomaceous earth; metakaolin; and olivine.

41.-42. (canceled)

43. The method of claim 1, wherein the cementitious aggregate composition absorbs more carbon dioxide during its manufacture than is emitted.

44.-46. (canceled)

47. The method claim 1, wherein the CO₂ of step (a) is from industrial waste, from environmental sources, or from molecular capture.

48.-56. (canceled)

57. The method of claim 1, wherein the cementitious aggregate composition comprises more CO₂ per gram than a standard cement.

58. (canceled)

59. The method of claim 1, wherein the cementitious aggregate composition comprises more calcium, silicon, aluminum, magnesium and/or iron per gram than a standard cement.

60.-74. (canceled)

75. The method of claim 1, wherein the cementitious aggregate composition does not substantially comprise MgO obtained from a calcination reaction.

76.-77. (canceled)

78. The method of claim 2, wherein the pressure in step (b) is from about ambient (1 bar) to about 250 bar.

79.-83. (canceled)

84. The method of claim 1, wherein sand, gravel, lightweight aggregate, or crushed stone, and a combination thereof is added to the combination of step (a).

85. (canceled)

86. The method of claim 1, wherein the latently hydraulic material is a slag.

87.-90. (canceled)

91. The method of claim 1, wherein the cementitious aggregate composition is molded into a shaped cementitious composition which permits attachment by flora and/or fauna.

92.-104. (canceled)

105. The method of claim 1, wherein the combination obtained in step (a) is selected from the group consisting of: (1) brine, (2) CO₂, and (3) a natural pozzolan; (1) brine, (2) CO₂, (3) a natural pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); (1) brine, (2) CO₂, and (3) a man-made pozzolan; (1) brine, (2) CO₂, (3) a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); (1) brine, (2) CO₂, and (3) a latently hydraulic material; (1) brine, (2) CO₂, (3) a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); (1) brine, (2) CO₂, and (3) a natural pozzolan and a man-made pozzolan; (1) brine, (2) CO₂, (3) a natural pozzolan and a man-made pozzolan, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); (1) brine, (2) CO₂, and (3) a natural pozzolan and a latently hydraulic material; (1) brine, (2) CO₂, (3) a natural pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); (1) brine, (2) CO₂, and (3) a man-made pozzolan and a latently hydraulic material; (1) brine, (2) CO₂, (3) a man-made pozzolan and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO); (1) brine, (2) CO₂, and (3) a natural pozzolan, a man-made pozzolan, and a latently hydraulic material; and (1) brine, (2) CO₂, (3) a natural pozzolan, a man-made pozzolan, and a latently hydraulic material, and (4) an activator (which may be any sodium, lithium or potassium salt or hydroxide and any alkali or alkali earth metal ions and including Mg(OH)₂, MgO, Ca(OH)₂, CaCO₃, Al₂(SO₄)₃, and/or CaO.

106.-118. (canceled)

119. A cementitious aggregate composition obtained by the method of claim 1.

120. (canceled)

121. A shaped cementitious composition obtained by the method of claim 91.

122.-128. (canceled)

129. A method for providing substrate for aquatic flora and/or fauna attachment, the method comprising depositing the shaped cementitious composition of claim 121 into a natural or artificial body of water which comprises aquatic flora and/or fauna in need of an attachment.

130.-135. (canceled)
